(19) United States
(12) Patent Application Publication
(10) Pub. No.: US 2025/0388941 A1
(43) Pub. Date: Dec. 25, 2025

(54) POLYPEPTIDE, OXYGENASE, AND APPLICATION THEREOF

(71) Applicants: AMANO ENZYME INC., Nagoya-shi (JP); NATIONAL INSTITUTE OF ADVANCED INDUSTRIAL SCIENCE AND TECHNOLOGY, Tokyo (JP); Kyoto University, Kyoto-shi (JP)

(72) Inventors: Satoru Ishihara, Kakamigahara-shi (JP); Satoshi Koikeda, Kakamigahara-shi (JP); Tomoshi Kameda, Tsukuba-shi (JP); Kimiyoshi Ikebe, Tsukuba-shi (JP); Yoichi Kurumida, Tsukuba-shi (JP); Jun Ogawa, Kyoto-shi (JP); Ryotaro Hara, Kyoto-shi (JP); Shigenobu Kishino, Kyoto-shi (JP); Michiki Takeuchi, Kyoto-shi (JP); Akinori Ando, Kyoto-shi (JP)

(73) Assignees: AMANO ENZYME INC., Nagoya-shi (JP); NATIONAL INSTITUTE OF ADVANCED INDUSTRIAL SCIENCE AND TECHNOLOGY, Tokyo (JP); Kyoto University, Kyoto-shi (JP)

(21) Appl. No.: 18/878,514

(22) PCT Filed: Jun. 26, 2023
(86) PCT No.: PCT/JP2023/023517

§ 371 Date: Dec. 23, 2024

(30) Foreign Application Priority Data
Jun. 27, 2022 (JP) 2022-102307

(51) Int. Cl. C12P 17/02; C12N 9/02; C12P 7/6432; C12P 7/6434

(52) U.S. Cl. CPC C12P 17/02; C12N 9/0071; C12P 7/6432; C12P 7/6434; C12Y 114/14001

(57) ABSTRACT

It is an object of the present invention to provide a modified oxygenase that specifically epoxidizes only the position-ω3 of highly unsaturated fatty acid. The present invention relates to: a polypeptide consisting of an amino acid sequence containing one or two or more amino acid substitutions selected from the group consisting of F87K/I/H, A330V, P25L and T438M, in the amino acid sequence as set forth in SEQ ID No: 2; and a polypeptide substantially identical to the aforementioned polypeptide.

### TECHNICAL FIELD

The present invention relates to a polypeptide, an oxygenase, and application thereof. Specifically, the present invention relates to an oxygenase obtained by modifying cytochrome P450 monooxygenase, and application thereof.

### BACKGROUND ART

Cytochrome P450 monooxygenase (hereinafter also referred to as “P450”) is a monooxygenase enzyme possessed by a wide range of organism species, ranging from bacteria to plants and mammals. Bacterium-derived P450 has been known to have a higher catalytic activity than eukaryote-derived P450. As such bacterium-derived P450, P450cam derived from Pseudomonas aeruginosa, P450-BM3 derived from Bacillus megaterium, which is one type of bacterium of genus Bacillus, and the like have been known.

Bacterium-derived P450 has a high catalytic activity of epoxidizing (hydroxylating) substrates such as fatty acids. In addition, since bacterium-derived P450 has a relatively high affinity to water, it is easy to acquire and handle the bacterium-derived P450. For these reasons, P450 is suitable as a biocatalyst. However, wild-type P450 does not necessarily have a high catalytic activity for epoxidizing industrially useful hydrocarbon substrates. Thus, various mutant forms of P450 have been proposed (for example, Patent Documents 1 to 3 and Non-Patent Documents 1 to 4).

Highly unsaturated fatty acids such as arachidonic acid, eicosapentaenoic acid, and docosahexaenoic acid are metabolized into various compounds in a living body and exhibit various physiological actions in individual tissues. For example, a cyclooxygenase system, a lipoxygenase system, and a P450 system are known as metabolic systems of arachidonic acid, and arachidonic acid is metabolized into many compounds in these systems. Among these metabolic systems, in the P450 system, the double bond of highly unsaturated fatty acid is epoxidized. In particular, a highly unsaturated fatty acid metabolite generated by epoxidizing the position-ω3 of highly unsaturated fatty acid is an important substance for anti-inflammatory action and anti-allergic action in a living body (Non-Patent Document 5).

Bacterium-derived P450 reacts with highly unsaturated fatty acid and thereby catalyzes the epoxidation of the position-ω3 even outside of a living body (Non-Patent Document 6). On the other hand, a method of epoxidizing highly unsaturated fatty acid utilizing a chemical oxidation reaction has also been known. In this case, a method of reacting with hydrogen peroxide in the presence of lipase has been reported (Patent Document 4).

### PRIOR ART DOCUMENTS

### Patent Documents

Patent Document 1: JP Patent Publication No. 2000-508163 A
Patent Document 2: JP Patent Publication No. 2003-517815 A
Patent Document 3: JP Patent Publication No. 2003-521889 A
Patent Document 4: JP Patent Publication No. 05-059032 A (1993)

### Non-Patent Documents

Non-Patent Document 1: Advanced Synthesis & Catalysis, 2006, 348.6:763-772.
Non-Patent Document 2: Drug metabolism and disposition, 2010, 38.5:732-739.
Non-Patent Document 3: Applied microbiology and biotechnology, 2011, 90.1:147-157
Non-Patent Document 4: International Journal of Molecular Sciences, 2021, 22.21:11380
Non-Patent Document 5: J Scientific reports, 2015, 5.1:1-8.
Non-Patent Document 6: Journal of Biological Chemistry, 1996, 271.37:22663-22671.

### SUMMARY OF INVENTION

### Objects to be Solved by the Invention

As mentioned above, a method for epoxidizing highly unsaturated fatty acid utilizing a chemical oxidation reaction has been known. According to this method, however, the double bonds at the positions-ω6 and -ω9 can be specifically epoxidized, but a highly unsaturated fatty acid metabolite, in which the physiologically important position-ω3 is epoxidized, cannot be produced.

Moreover, a method for epoxidizing the position-ω3 of highly unsaturated fatty acid using bacterium-derived P450 has also been known. However, in the studies of the present inventors, it has been found that the reaction position specificity of the wild-type bacterium-derived P450 is low, and as the reaction progresses, not only the epoxidation of the position-ω3, but also the epoxidation of the double bonds at other positions progresses, and that finally the fatty acid is decomposed. Thus, even in the case of using the bacterium-derived P450, it has been difficult to produce a highly unsaturated fatty acid metabolite, in which only the physiologically important position-ω3 has been specifically epoxidized.

Therefore, it is an object of the present invention to provide a modified oxygenase that specifically epoxidizes only the position-ω3 of highly unsaturated fatty acid.

### Means for Solving the Objects

As a result of intensive studies directed towards achieving the aforementioned object, the present inventors have introduced a specific mutation into a predetermined position in the amino acid sequence of an oxygenase, and have successfully obtained a modified oxygenase that produces ω3-epoxidized fatty acid, in which only the position-ω3 of highly unsaturated fatty acid is epoxidized.

Specifically, the present invention has the following configurations.
[1] A polypeptide described in any of the following (1) to (3):
   (1) a polypeptide consisting of an amino acid sequence containing one or two or more amino acid substitutions selected from the group consisting of F87K/I/H, A330V, P25L and T438M, in the amino acid sequence as set forth in SEQ ID No: 2;
   (2) a polypeptide containing a substitution, addition, insertion or deletion of one or several amino acid residues in the amino acid sequence described in the above (1), having a catalytic activity of epoxidizing the position-ω3 of ω3 fatty acid, and retaining one or two or more amino acid substitutions selected from the group consisting of F87K/I/H, A330V, P25L and T438M; and
   (3) a polypeptide having a sequence identity of 90% or more to the amino acid sequence described in the above (1), having a catalytic activity of epoxidizing the position-ω3 of ω3 fatty acid, and retaining one or two or more amino acid substitutions selected from the group consisting of F87K/I/H, A330V, P25L and T438M,
   (provided that F87K/I/H indicates that F, the amino acid residue at position 87 in SEQ ID No: 2, is substituted with K, I or H; A330V indicates that A, the amino acid residue at position 330 in SEQ ID No: 2, is substituted with V; P25L indicates that P, the amino acid residue at position 25 in SEQ ID No: 2, is substituted with L; and T438M indicates that T, the amino acid residue at position 438 in SEQ ID No: 2, is substituted with M).
[2] The polypeptide according to [1], in which the sequence identity in the above (3) is 95% or more.
[3] The polypeptide according to [1] or [2], which is an oxygenase.
[4] An enzyme agent for epoxidation of the position-ω3 of ω3 fatty acid, containing the polypeptide according to any one of [1] to [3].
[5] The enzyme agent for epoxidation of the position-ω3 according to [4], further containing one or two or more enzymes selected from the group consisting of a NADPH-regenerating enzyme, an active oxygen-removing enzyme, and a hydrogen peroxide-removing enzyme.
[6] DNA described in any of the following (1) to (3):
   (1) DNA encoding the polypeptide according to [1];
   (2) DNA consisting of the nucleotide sequence as set forth in any of SEQ ID Nos: 3, 5, 7 and 9; and
   (3) DNA containing a sequence equivalent to the nucleotide sequence as set forth in any of SEQ ID Nos: 3, 5, 7 and 9, and encoding a polypeptide having a catalytic activity of epoxidizing the position-ω3 of ω3 fatty acid.
[7] An expression cassette, containing the DNA according to [6].
[8] A recombinant vector, containing the DNA according to [6].
[9] A transformant, having the DNA according to [6], the expression cassette according to [7], or the recombinant vector according to [8].
[10] A method for producing an oxygenase, including
   culturing the transformant according to [9].
[11] A method for producing ω3-epoxidized fatty acid, including
   allowing the polypeptide according to any one of [1] to [3] or the enzyme agent for epoxidation of the position-ω3 according to [4] or [5] to act on ω3 fatty acid.
[12] A method for epoxidizing ω3 fatty acid, including
   allowing the polypeptide according to any one of [1] to [3] or the enzyme agent for epoxidation of the position-ω3 according to [4] or [5] to act on ω3 fatty acid.
[13] A composition, containing the ω3-epoxidized fatty acid, which is obtained by the production method according to [11].

### Advantageous Effects of Invention

According to the present invention, there can be obtained a modified oxygenase that specifically epoxidizes only the position-ω3 of highly unsaturated fatty acid. In addition, according to the present invention, there are provided a method for producing ω3-epoxidized fatty acid, in which only the position-ω3 is epoxidized, and ω3-epoxidized fatty acid produced by the aforementioned production method.

### BRIEF DESCRIPTION OF DRAWINGS

FIG. 1 shows the analysis results of HPLC analysis of the detection amount of 17,18-EpETE (17,18-epoxy eicosatetraenoic acid) generated from EPA (eicosapentaenoic acid), which is a substrate, when mutant enzymes (F87K, F87I, and F87H) are used.

FIG. 2 shows the analysis results of HPLC analysis of epoxidized compounds generated from EPA (eicosapentaenoic acid), which is a substrate.

FIG. 3 shows the analysis results of HPLC analysis of epoxidized compounds generated from EPA (eicosapentaenoic acid), which is a substrate.

FIG. 4 shows the analysis results of HPLC analysis of epoxidized compounds generated from EPA (eicosapentaenoic acid), which is a substrate.

FIG. 5 shows the analysis results of HPLC analysis of epoxidized compounds generated from EPA (eicosapentaenoic acid), which is a substrate.

FIG. 6 shows the analysis results of HPLC analysis of epoxidized compounds generated from DHA (docosahexaenoic acid), which is a substrate.

FIG. 7 shows the analysis results of HPLC analysis of epoxidized compounds generated from DHA (docosahexaenoic acid), which is a substrate.

FIG. 8 shows the analysis results of HPLC analysis of epoxidized compounds generated from DHA (docosahexaenoic acid), which is a substrate.

FIG. 9 shows the analysis results of HPLC analysis of epoxidized compounds generated from DPA (docosapentaenoic acid), which is a substrate.

FIG. 10 shows the analysis results of HPLC analysis of epoxidized compounds generated from DPA (docosapentaenoic acid), which is a substrate.

FIG. 11 shows the analysis results of HPLC analysis of epoxidized compounds generated from DPA (docosapentaenoic acid), which is a substrate.

### EMBODIMENT OF CARRYING OUT THE INVENTION

Hereafter, the present invention will be described in detail. The configuration requirements described below may be described based on representative embodiments or specific examples, but the present invention is not limited to such embodiments.

In the present description, the 20 types of amino acid residues in the amino acid sequences may be expressed by one-letter abbreviations in some cases. In that case, glycine (Gly) is G, alanine (Ala) is A, valine (Val) is V, leucine (Leu) is L, isoleucine (Ile) is I, phenylalanine (Phe) is F, tyrosine (Tyr) is Y, tryptophan (Trp) is W, serine (Ser) is S, threonine (Thr) is T, cysteine (Cys) is C, methionine (Met) is M, Aspartic acid (Asp) is D, glutamic acid (Glu) is E, asparagine (Asn) is N, glutamine (Gln) is Q, lysine (Lys) is K, arginine (Arg) is R, histidine (His) is H, and proline (Pro) is P. Moreover, in the present description, in the amino acid sequences displayed, the N-terminus is at the left end and the C-terminus is at the right end.

In the present description, “non-polar amino acids” include alanine, valine, leucine, isoleucine, proline, methionine, phenylalanine and tryptophan. “Uncharged amino acids” include glycine, serine, threonine, cysteine, tyrosine, asparagine and glutamine. “Acidic amino acids” include aspartic acid and glutamic acid. “Basic amino acids” include lysine, arginine and histidine.

In the present description, “substitution” refers not only to the case where amino acid residue substitution is artificially introduced, but also to the case where amino acid residue substitution is introduced naturally, that is, the case where the amino acid residue was originally different. In the present description, the substitution of amino acid residues may be an artificial substitution, or a natural substitution, but an artificial substitution is preferable.

### (Polypeptide(Oxygenase))

The present invention relates to a polypeptide described in any of the following (1) to (3):
(1) a polypeptide consisting of an amino acid sequence containing one or two or more amino acid substitutions selected from the group consisting of F87K/I/H, A330V, P25L and T438M, in the amino acid sequence as set forth in SEQ ID No: 2;
(2) a polypeptide containing a substitution, addition, insertion or deletion of one or several amino acid residues in the amino acid sequence described in the above (1), having a catalytic activity of epoxidizing the position-ω3 of ω3 fatty acid, and retaining one or two or more amino acid substitutions selected from the group consisting of F87K/I/H, A330V, P25L and T438M; and
(3) a polypeptide having a sequence identity of 90% or more to the amino acid sequence described in the above (1), having a catalytic activity of epoxidizing the position-ω3 of ω3 fatty acid, and retaining one or two or more amino acid substitutions selected from the group consisting of F87K/I/H, A330V, P25L and T438M.

In the above context, F87K/I/H indicates that F, the amino acid residue at position 87 in SEQ ID No: 2, is substituted with K, I or H; A330V indicates that A, the amino acid residue at position 330 in SEQ ID No: 2, is substituted with V; P25L indicates that P, the amino acid residue at position 25 in SEQ ID No: 2, is substituted with L; and T438M indicates that T, the amino acid residue at position 438 in SEQ ID No: 2, is substituted with M.

It is to be noted that the polypeptides of the above (1) to (3) include not only polypeptides obtained by artificial substitution, but also naturally substituted polypeptides that have such amino acid sequences.

In the present description, an amino acid substitutions is expressed as a combination of a one-letter abbreviation representing the amino acid residue before substitution, a number representing the position of an amino acid residue in which the amino acid substitution occurs (the position from the N-terminal side in a specific amino acid sequence), and a one-letter abbreviation representing the amino acid residue after substitution. For example, if the phenylalanine at position 87 is substituted with lysine, it is expressed as “F87K.” In addition, if the amino acid residue after substitution may be any of several types, the one-letter abbreviation representing the amino acid residue after substitution is written together with the symbol “/”. For example, F87K/I/H indicates that the phenylalanine at position 87 is substituted with lysine, isoleucine, or histidine.

Moreover, when a combination (combined use) of two or more substitutions is expressed, the symbol “-” is used. For example, a combination of substitution of the phenylalanine at position 87 with lysine and substitution of the alanine at position 330 with valine is expressed as “F87K-A330V.”

The polypeptide described in any one of the above (1) to (3) has a catalytic activity of specifically epoxidizing the position-ω3 of highly unsaturated fatty acid (ω3 fatty acid), more than the polypeptide of SEQ ID No: 2. The present inventors have designed an enzyme by performing molecular reaction simulation of an enzyme-substrate reaction based on molecular dynamics (MD) calculation from the three-dimensional structure information of P450, and have produced, cultured, and evaluated a mutant recombinant P450 based on the design. As a result, the present inventors have succeeded in obtaining a modified oxygenase (P450) with improved specificity for epoxidation of the position-ω3.

The polypeptide of SEQ ID No: 2 is a cytochrome P450 monooxygenase derived from Bacillus megaterium. The polypeptide of the present invention is preferably a modified oxygenase, more preferably a modified monooxygenase, and further preferably a modified monooxygenase derived from a cytochrome P450 monooxygenase derived from Bacillus megaterium. Herein, the monooxygenase is an enzyme that has a catalytic action of introducing one oxygen atom into a compound using an oxygen molecule as a substrate, and requires heme, non-heme iron or a metal ion as a cofactor. It is to be noted that such a monooxygenase may have other functions, as long as the above-described function is its main action.

The polypeptide of the present invention may be a part of an oxygenase, and further, the polypeptide or the oxygenase may be a part of a larger protein (e.g., a fusion protein). Examples of a sequence added to a fusion protein may include a sequence that is useful for purification, such as multiple histidine residues, and an additional sequence that ensures stability during recombinant production.

The polypeptide of the present invention consists of an amino acid sequence containing one or two or more amino acid substitutions selected from, at least, the group consisting of F87K/I/H, A330V, P25L and T438M, in the amino acid sequence as set forth in SEQ ID No: 2. The polypeptide of the present invention preferably contains, at least, an amino acid substitution of F87K, F87I or F87H, more preferably contains an amino acid substitution of F87K or F87I, and particularly preferably contains an amino acid substitution of F87K.

The polypeptide of the present invention preferably consists of an amino acid sequence containing an amino acid substitution of F87K/I/H and one or two or more amino acid substitutions selected from the group consisting of A330V, P25L and T438M, in the amino acid sequence as set forth in SEQ ID No: 2. A more preferred aspect is a polypeptide consisting of amino acid sequence containing an amino acid substitution of F87K and one or two or more amino acid substitutions selected from the group consisting of A330V, P25L and T438M, in the amino acid sequence as set forth in SEQ ID No: 2. Specific aspects may include a polypeptide consisting of an amino acid sequence containing an amino acid substitution of F87K-A330V, a polypeptide consisting of an amino acid sequence containing amino acid substitutions of F87K-P25L, a polypeptide consisting of an amino acid sequence containing amino acid substitutions of F87K-T438M, a polypeptide consisting of an amino acid sequence containing amino acid substitutions of F87K-A330V-P25L, a polypeptide consisting of an amino acid sequence containing amino acid substitutions of F87K-A330V-T438M, a polypeptide consisting of an amino acid sequence containing amino acid substitutions of F87K-P25L-T438M, and a polypeptide consisting of an amino acid sequence containing amino acid substitutions of F87K-A330V-P25L-T438M, in the amino acid sequence as set forth in SEQ ID No: 2. Among others, preferable is a polypeptide consisting of an amino acid sequence containing, at least, double amino acid substitutions at F87K and A330V in the amino acid sequence as set forth in SEQ ID No: 2. Furthermore, a polypeptide consisting of an amino acid sequence containing triple amino acid substitutions, having a mutation at P25L or T438M in addition to the double mutations at F87K and A330V, is also preferable; and a polypeptide consisting of an amino acid sequence containing amino acid substitutions of F87K-A330V-T438M is particularly preferable because it is more likely to exhibit a catalytic activity of specifically epoxidizing the ω3 position.

In the present description, in the polypeptides of the above (2) and (3), the amino acid residues other than those at positions 87, 330, 25, and 438 of SEQ ID No: 2 may be referred to as “optional difference sites.” In the present description, the “optional difference sites” are sites in which differences are permitted as long as they do not significantly affect the properties of the polypeptide. A polypeptide that has a difference in the amino acid sequence at an optional difference site but has a catalytic activity of specifically epoxidizing the position-ω3 of ω3 fatty acid, which is improved compared to the polypeptide consisting of the amino acid sequence as set forth in SEQ ID No: 2, is referred to as a different body of the polypeptide of the above (1). In other words, the polypeptides (2) and (3) above are different bodies of the polypeptide of the above (1). In addition, it is preferable that such a different body of the polypeptide has a difference in the amino acid sequence at an optional difference site compared to the polypeptide of the above (1), but has the properties of the polypeptide that are substantially identical to the polypeptide of the above (1). Besides, the phrase “the properties of the polypeptide that are substantially identical to . . . ” means that the catalytic activity of specifically epoxidizing the position-ω3 of ω3 fatty acid is equivalent.

In the polypeptide of the above (2), the modification of an amino acid(s) introduced into the optional difference site may be one type of modification (for example, a substitution only) selected from a substitution, an addition, an insertion, and a deletion, or may also be two or more types of modifications (for example, a substitution and an insertion). In the polypeptide of the above (2), the number of amino acid differences at the optional difference site may be one or several amino acids. The number of differences may be, for example, 1 to 80, 1 to 70, 1 to 60, 1 to 50, 1 to 40, 1 to 30, 1 to 20, 1 to 10, 1 to 8, 1 to 7, 1 to 6, 1 to 5, 1 to 4, 1 to 3, or 1 or 2.

The polypeptide of the above (3) has a sequence identity of preferably 60% or more, more preferably 70% or more, even more preferably 80% or more, further preferably 90% or more, still further preferably 95% or more, particularly preferably 97% or more, and most preferably 99% or more, to the amino acid sequence described in the above (1). Herein, as for the polypeptide of the above (3), the sequence identity to the amino acid sequence described in the above (1) means a sequence identity calculated by comparing with the amino acid sequence described in the above (1). In the present description, the term “sequence identity” refers to the identity value of the amino acid sequence, which is obtained by the b12seq program (Tatiana A. Tatsusova, Thomas L. Madden, FEMS Microbiol. Lett., Vol. 174, pp. 247-250, 1999) of BLASTPACKAGE [sgi32 bit edition, Version 2.0.12; available from National Center for Biotechnology Information (NCBI)]. The parameters are set to Gap insertion Cost value: 11, Gap extension Cost value: 1 for calculation.

When an amino acid substitution is introduced into the optional difference site of the polypeptides of the above (2) and (3), one preferred aspect of the amino acid substitution to be introduced may be a conservative substitution. Examples of the amino acid substitution in the polypeptides of the above (2) and (3) may include: a substitution with another nonpolar amino acid if the amino acid before substitution is a nonpolar amino acid; a substitution with another uncharged amino acid if the amino acid before substitution is an uncharged amino acid; a substitution with another acidic amino acid if the amino acid before substitution is an acidic amino acid; and a substitution with another basic amino acid if the amino acid before substitution is a basic amino acid.

Besides, in the polypeptides of the above (2) and (3), since the amino acids at positions 267 (glutamic acid), 268 (threonine), 264 (alanine), and 393 (phenylalanine) in the amino acid sequence as set forth in SEQ ID No: 2 play the role of electron transfer and oxygen binding, and are considered to be important residues for active catalysis, it is desirable not to introduce substitutions or deletions into these positions.

In the polypeptides of the above (2) and (3), the “catalytic activity of epoxidizing the position-ω3 of ω3 fatty acid” can be evaluated by detecting ω3-epoxidized fatty acid obtained by epoxidizing only the substrate, namely, the position-ω3 of ω3 fatty acid, according to column chromatography, etc. Specifically, when the production percentage of ω3-epoxidized fatty acid increases compared to the case where the polypeptide consisting of the amino acid sequence as set forth in SEQ ID No: 2 is used, it can be said that there is a “catalytic activity of epoxidizing the position-ω3 of ω3 fatty acid.” In addition, the production percentage of ω3-epoxidized fatty acid when the polypeptides of the above (2) and (3) are used is preferably equal to or higher than the production percentage of ω3-epoxidized fatty acid when the polypeptide of the above (1) is used. For example, when the production percentage of ω3-epoxidized fatty acid when the polypeptide of the above (1) is used is set to be 1, the production percentage of ω3-epoxidized fatty acid when the polypeptides of the above (2) and (3) are used is preferably 0.8 to 1.2, more preferably 0.9 to 1.1, and further preferably 0.95 to 1.05. Besides, the production percentage of ω3-epoxidized fatty acid means the ratio of the mass (molar concentration) of ω3-epoxidized fatty acid to the total mass (total molar concentration) of a product obtained by allowing a polypeptide (oxygenase) to act on ω3 fatty acid serving as a substrate.

The polypeptide (oxygenase) of the present invention has high substrate specificity and specifically epoxidizes the position-ω3 of ω3 fatty acid. Specifically, in a case where the production percentage of ω3-epoxidized fatty acid is 40% or more when the polypeptides of the above (1) to (3) are each allowed to act on eicosapentaenoic acid (EPA) used as a substrate, it can be determined that the position-ω3 of ω3 fatty acid is specifically epoxidized. Besides, the production percentage of ω3-epoxidized fatty acid that is obtained when the polypeptides of the above (1) to (3) are each allowed to act on EPA is preferably 40% or more, more preferably 50% or more, even more preferably 60% or more, further preferably 65% or more, still further preferably 70% or more, particularly preferably 80% or more, and most preferably 85% or more. Moreover, the production percentage of ω3-epoxidized fatty acid obtained when the polypeptides of the above (1) to (3) are each allowed to act on EPA may also be 100%.

### (Enzyme Agent)

The present invention may relate to an enzyme agent for epoxidation of the position-ω3 of highly unsaturated fatty acid (ω3 fatty acid), containing the above-mentioned polypeptide. The enzyme agent for epoxidation of the position-ω3 contains the above-mentioned polypeptide as an active ingredient. Besides, the enzyme agent may consist of the above-mentioned polypeptide.

The content of the above-mentioned polypeptide in the enzyme agent of the present invention is not particularly limited, and it can be appropriately set within a range in which a catalytic activity of epoxidizing the position-ω3 of ω3 fatty acid is exhibited.

The enzyme agent of the present invention may contain other components, in addition to the above-mentioned polypeptide, to such an extent that they do not affect the effects of the present invention. Examples of other components may include other enzymes other than the above-mentioned polypeptide, additives, and culture residues generated by the production method as described below. Among them, it is preferable that the enzyme agent contains other enzymes other than the above-mentioned polypeptide, and when the position-ω3 of ω3 fatty acid is epoxidized, an enzyme to be used in combination may be used in addition to the above-mentioned polypeptide.

Specifically, the enzyme agent may contain, in addition to the above-mentioned polypeptide, one or two or more enzymes selected from the group consisting of a NADPH-regenerating enzyme, an active oxygen-removing enzyme, and a hydrogen peroxide-removing enzyme. When the position-ω3 of ω3 fatty acid is epoxidized, the above-mentioned combined-use enzyme is used in addition to the above-mentioned polypeptide, so that the reaction rate can be increased and the production rate of ω3-epoxidized fatty acid can be increased.

The NADPH-regenerating enzyme is not particularly limited, as long as it is an enzyme capable of converting the coenzyme NADP to NADPH. The NADPH-regenerating enzyme realizes reactivation of the enzyme by regenerating the coenzyme NADPH consumed by monooxygenase in the reaction. Therefore, it has the effect of improving the substrate-converting speed when compared at the same reaction time.

The types of NADPH-regenerating enzymes may include glucose dehydrogenase, alcohol dehydrogenase, D-lactate dehydrogenase, malate dehydrogenase, isocitrate dehydrogenase, and glucose-6-phosphate dehydrogenase. As such a NADPH-regenerating enzyme, one type from these NADPH-regenerating enzymes may be used, or two or more types of these enzymes may also be used in combination. Among these NADPH-regenerating enzymes, NAD (P)-dependent glucose dehydrogenase (glucose dehydrogenase (NAD)) is preferable from the viewpoint of further enhancing the effects. A specific example of the glucose dehydrogenase may be glucose dehydrogenase derived from Bacillus megaterium.

The NADPH-regenerating enzyme can be prepared by a known method. As an example, in the case of preparing glucose dehydrogenase derived from Bacillus megaterium, the glucose dehydrogenase can be easily prepared by a method of culturing a producing bacterium and separating glucose dehydrogenase using a known mean, a method of using a genetic recombination technique, and the like. Otherwise, a commercially available product may be used as such a NADPH-regenerating enzyme. Examples of the commercially available NADPH-regenerating enzyme product may include Glucose Dehydrogenase manufactured by Amano Enzyme Inc., and ADH, rD-LDH, rMDH, rICDH and rG6PDH manufactured by Oriental Yeast Co., Ltd.

The active oxygen-removing enzyme and the hydrogen peroxide-removing enzyme are not particularly limited, as long as they are enzymes that disproportionate active oxygen into hydrogen peroxide and molecular oxygen, or that can convert hydrogen peroxide into water. The active oxygen-removing enzyme and the hydrogen peroxide-removing enzyme have the effect of decomposing active oxygen and hydrogen peroxide that are generated by monooxygenase during the reaction, thereby protecting enzymes. Accordingly, these enzymes have the effect of preventing inactivation of monooxygenase and improving the substrate-converting speed.

The types of the active oxygen-removing enzyme and the hydrogen peroxide-removing enzyme may include catalase, peroxidase, and superoxide dismutase. One type from these active oxygen- or hydrogen peroxide-removing enzymes may be used, or two or more types of these enzymes may also be used in combination. Among these active oxygen- or hydrogen peroxide-removing enzymes, catalase and superoxide dismutase are preferable from the viewpoint of further enhancing the effects.

As such an active oxygen-removing enzyme and a hydrogen peroxide-removing enzyme, catalase, peroxidase, and superoxide dismutase, which are derived from animal organs, horseradish, Escherichia coli, and the genus Aspergillus, can also be used. Among these catalases, peroxidases, and superoxide dismutases, catalase derived from animal organs or superoxide dismutase derived from animals are preferable from the viewpoint of further enhancing the effects.

The active oxygen-removing enzyme and the hydrogen peroxide-removing enzyme can be prepared by known methods. For example, when animal-derived catalase is prepared, it can be easily prepared by a method of separating catalase from animal organs using a known means, a method of using a genetic recombination technique, and the like. Otherwise, as such active oxygen- or hydrogen peroxide-removing enzymes, commercially available products may be used. Examples of the commercially available active oxygen- or hydrogen peroxide-removing enzyme products may include Catalase manufactured by FUJIFILM Wako Pure Chemical Corporation, Peroxidase manufactured by FUJIFILM Wako Pure Chemical Corporation, Superoxide Dismutase manufactured by Sigma-Aldrich, and Superoxide Dismutase manufactured by Toyobo Co., Ltd.

Examples of other enzymes that may be contained in the enzyme agent may include amylase (α-amylase, β-amylase, and glucoamylase), glucosidase (α-glucosidase and β-glucosidase), galactosidase (α-galactosidase and β-galactosidase), protease (acid protease, neutral protease, and alkaline protease), peptidase (leucine peptidase and aminopeptidase), lipase, esterase, cellulase, phosphatase (acid phosphatase and alkaline phosphatase), nuclease, deaminase, oxidase, dehydrogenase, glutaminase, pectinase, catalase, dextranase, transglutaminase, protein deamidase, pullulanase, peroxidase, and superoxide dismutase. These other enzymes may be one type or multiple types.

Examples of the additives may include an excipient, a buffer agent, a suspending agent, a stabilizer, a preservative, an antiseptic, and a normal saline. Examples of the excipient may include starch, dextrin, maltose, trehalose, lactose, D-glucose, sorbitol, D-mannitol, white sugar, and glycerol. Examples of the buffer agent may include phosphate, citrate, and acetate. Examples of the stabilizer may include propylene glycol and ascorbic acid. Examples of the preservative may include phenol, benzalkonium chloride, benzyl alcohol, chlorobutanol, and methylparaben. Examples of the antiseptic may include ethanol, benzalkonium chloride, paraoxybenzoic acid, and chlorobutanol. These additives may be one type or multiple types.

Examples of culture residues may include components derived from the medium, contaminant proteins, and bacterial components.

The form of the enzyme agent of the present invention is not particularly limited, and examples thereof may include a liquid form, a solid form (powders, granules, etc.), and a form immobilized on a carrier.

### (DNA)

The present invention relates to DNA described in any of the following (1) to (3):
(1) DNA encoding the above-mentioned polypeptide;
(2) DNA consisting of the nucleotide sequence as set forth in any of SEQ ID Nos: 3, 5, 7 and 9; and
(3) DNA containing a sequence equivalent to the nucleotide sequence as set forth in any of SEQ ID Nos: 3, 5, 7 and 9, and encoding a polypeptide having a catalytic activity of epoxidizing the position-ω3 of ω3 fatty acid.

The nucleotide sequence as set forth in SEQ ID No: 3 is the cDNA sequence of the gene encoding the polypeptide consisting of the amino acid sequence of the F87K mutant shown in SEQ ID No: 4; the nucleotide sequence as set forth in SEQ ID No: 5 is the cDNA sequence of the gene encoding the polypeptide consisting of the amino acid sequence of the F87K and A330V double mutant shown in SEQ ID No: 6; the nucleotide sequence as set forth in SEQ ID No: 7 is the cDNA sequence of the gene encoding the polypeptide consisting of the amino acid sequence of the P25L, F87K and A330V triple mutant shown in SEQ ID No: 8; and the nucleotide sequence as set forth in SEQ ID No: 9 is the cDNA sequence of the gene encoding the polypeptide consisting of the amino acid sequence of the F87K, A330V and T438M triple mutant shown in SEQ ID No: 10.

The homology of the DNA of the above (3) to the DNA of the above (1) is preferably 75% or more, more preferably 80% or more, even more preferably 85% or more, further preferably 90% or more, still further preferably 95% or more, and particularly preferably 99% or more. Even if certain DNA has a sequence different from the nucleotide sequence as set forth in any of SEQ ID Nos: 3, 5, 7 and 9, it can be said that the certain DNA is equivalent to the nucleotide sequence as set forth in any of SEQ ID Nos: 3, 5, 7 and 9, so long as it encodes a polypeptide having a catalytic activity of specifically epoxidizing the position-ω3 of ω3 fatty acid, and that the certain DNA is included in the DNA of the present invention.

The “homology” of DNA is calculated using publicly disclosed or commercially available software having an algorithm of comparing the sequence with a reference sequence used as a query sequence. Specifically, BLAST, FASTA, GENETYX (manufactured by Fukuoka Software Development Co., Ltd.) or the like can be used, and these may be used with the default parameters set.

Moreover, a nucleotide sequence encoding a polypeptide, in which one or several amino acid residues are substituted, added, inserted or deleted at an optional difference site in the amino acid sequence, is also included in the DNA of the present invention, as long as it encodes a polypeptide having a catalytic activity of specifically epoxidizing the position-ω3 of ω3 fatty acid.

Furthermore, DNA that hybridizes under stringent conditions with DNA consisting of a nucleotide sequence complementary to the DNA consisting of the nucleotide sequence encoding the above-mentioned polypeptide is also included in the DNA of the present invention, as long as it encodes a polypeptide having a catalytic activity of specifically epoxidizing the position-ω3 of ω3 fatty acid. Herein, the stringent conditions include, for example, conditions in which a nylon membrane on which DNA is fixed is incubated with a probe at 65° C. for 20 hours in a solution containing 6×SSC (1×SSC is a solution of 8.76 g of sodium chloride and 4.41 g of sodium citrate dissolved in 1 L of water), 1% SDS, 100 μg/mL salmon sperm DNA, 0.1% bovine serum albumin, 0.1% polyvinylpyrrolidone, and 0.1% Ficoll (in the present description, “%” means “w/v”) for hybridization. However, the stringent conditions are not limited to these conditions, and a person skilled in the art can set the hybridization conditions by taking into consideration the conditions such as the salt concentration of the buffer solution and the temperature, and also, other conditions such as the probe concentration, the probe length, and the reaction time. For detailed procedures of the hybridization method, there can be referred to Molecular Cloning, A Laboratory Manual 2nd ed. (Cold Spring Harbor Laboratory Press (1989)), etc.

Hereafter, an example of a method for obtaining the DNA of the present invention by hybridization will be described. However, the method for obtaining the DNA of the present invention is not limited to the following method.

First, DNA obtained from an appropriate gene source is connected with a plasmid or a phage vector according to a usual method, so as to prepare a DNA library. This library is introduced into an appropriate host, and the obtained transformant is cultured on a plate. Then, grown colonies or plaques are transcribed onto a nitrocellulose or nylon membrane, and DNA is fixed on the membrane after a denaturation treatment. This membrane is incubated under the above-mentioned stringent conditions in a solution of the above-mentioned composition containing a probe previously labeled with 32P or the like, and hybridization is then performed. As such a probe, a polynucleotide encoding the entire or a part of the amino acid sequence as set forth in SEQ ID No: 2 can be used.

After completion of the hybridization, non-specifically adsorbed probes are washed away, and clones that have formed a hybrid with the probes are identified by autoradiography or the like. This operation is repeated until a hybrid-forming clone can be isolated. Finally, a gene encoding a protein having an enzyme activity of interest is selected from the obtained clones. Gene isolation can be performed by known polynucleotide extraction methods such as an alkaline method.

Moreover, the DNA of the present invention can also be isolated from a microorganism that produces the above predetermined polypeptide. For example, using the genomic DNA of Bacillus megaterium as a template, DNA of interest can be isolated from the genome of the above-mentioned microorganism by performing PCR or a hybridization method using primers or probes designed from known amino acid sequence information taking gene degeneracy into consideration, or using primers or probes designed based on known nucleotide sequence information.

The DNA of the present invention includes many types of DNAs derived from codon degeneracy. Many types of DNA encoding an identical amino acid sequence can be easily, artificially produced using known genetic engineering techniques. For example, in the production of a protein by genetic engineering, if the codon used on the original gene encoding the protein of interest is one that is used at low frequency in the host, the expression level of the protein may be low. In such a case, the codon usage frequency can be optimized for the host without changing the encoded amino acid sequence, thereby achieving high expression of the protein of interest.

As an index of codon usage frequency, the total of the host optimal codon usage frequency of each codon may be adopted. The optimal codon is defined to be a codon with the highest usage frequency among the codons corresponding to an identical amino acid. The codon usage frequency is not particularly limited, as long as it is optimized for the host. For example, the following are examples of optimal codons for E. coli.

F: phenylalanine (ttt), L: leucine (ctg), I: isoleucine (att), M: methionine (atg), V: valine (gtg), Y: tyrosine (tat), stop codon (taa), H: histidine (cat), Q: glutamine (cag), N: asparagine (aat), K: lysine (aaa), D: aspartic acid (gat), E: glutamic acid (gaa), S: serine (agc), P: proline (ccg), T: threonine (acc), A: alanine (gcg), C: cysteine (tgc), W: tryptophan (tgg), R: arginine (cgc), and G: glycine (ggc).

Examples of the method of introducing a mutation into a gene to artificially modify an amino acid sequence may include known methods such as a Kunkel method and a Gapped Duplex method, and the use of mutation introduction kits utilizing a site-specific mutagenesis method, such as QuikChange™ Site-Directed Mutagenesis Kit (Stratagene), GeneTailor™ Site-Directed Mutagenesis System (Invitrogen Co., Ltd.), and TaKaRa Site-Directed Mutagenesis System (Mutan-K, Mutan-Super Express Km, etc.: Takara Bio, Inc.).

The nucleotide sequence of DNA can be confirmed by sequencing using a common method. For example, the nucleotide sequence of DNA can be confirmed by a dideoxynucleotide chain termination method (Sanger et al. (1977) Proc. Natl. Acad. Sci. USA 74:5463), etc. In addition, the sequence can be analyzed utilizing an appropriate DNA Sequencer.

Whether or not the obtained DNA is DNA encoding a polypeptide of interest can be confirmed by comparing the determined nucleotide sequence with the nucleotide sequence as set forth in SEQ ID No: 1. Alternatively, such confirmation can also be carried out by comparing the amino acid sequence estimated from the determined nucleotide sequence with the amino acid sequence as set forth in SEQ ID No: 2. Otherwise, such confirmation can also be carried out by measuring the catalytic activity of the expressed polypeptide (i.e., the catalytic activity of specifically epoxidizing the position-ω3 of ω3 fatty acid).

### (Expression Cassette/Recombinant Vector)

The present invention may relate to an expression cassette containing the above-mentioned DNA, or a recombinant vector containing the above-mentioned DNA. The expression cassette or the recombinant vector of the present invention can be obtained by linking a promoter and a terminator to the above-mentioned DNA. In addition, the recombinant vector can be obtained by inserting the expression cassette of the present invention or the DNA of the present invention into an expression vector.

The expression cassette of the present invention or the recombinant vector of the present invention may contain, as control factors, transcription elements such as an enhancer, a CCAAT box, a TATA box, an SPI site, etc., as necessary, in addition to a promoter and a terminator. These control factors may be operably linked to the DNA of the present invention. The phrase “be operably linked to” means that various control factors that regulate the DNA of the present invention are linked to the DNA of the present invention in a state in which the factors can operate in host cells.

The expression vector for constructing the recombinant vector of the present invention is preferably an expression vector that is constructed for genetic recombination from a phage, a plasmid or a virus capable of autonomously replicating in a host. Such expression vectors are known, and examples of commercially available expression vectors may include pQE vectors (QIAGEN K.K.), pDR540 and pRIT2T (GE HealthCare Biosciences), pET vectors (Merck & Co.), and pH Y300PLK and pBE-S (Takara Bio, Inc.). As such an expression vector, a suitable combination with the host cells may be selected and used. For example, when Escherichia coli is used as host cells, examples of the combination of the expression vector and the host cells may include a combination of a pBAD vector and a DH5a E. coli strain, a combination of a pET vector and a BL21 (DE3) E. coli strain, and a pDR540 vector and a JM109 E. coli strain. The binding of DNA and an expression cassette or an expression vector is carried out, for example, using DNA ligase.

### (Transformant)

The present invention may relate to a transformant having the above-mentioned DNA and the above-mentioned expression cassette or the above-mentioned recombinant vector. In other words, the transformant of the present invention is a transformant obtained by transforming a host with an expression cassette containing the above-mentioned DNA, or a recombinant vector containing the above-mentioned DNA.

The host used for producing the transformant is not particularly limited, as long as it can express the traits of a gene containing the DNA of the present invention. It is preferable that the host enables introduction of a gene therein, can stably retain the expression cassette or the recombinant vector, and can grow autonomously. Suitable examples of the hosts may include: bacteria belonging to the genus Escherichia such as Escherichia coli, the genus Bacillus such as Bacillus subtilis, and the genus Pseudomonas such as Pseudomonas putida; and yeasts. However, animal cells, insect cells, plants, etc. may also be used as hosts.

The transformant of the present invention can be obtained by introducing an expression cassette containing the above-mentioned DNA or a recombinant vector containing the above-mentioned DNA into a host. The site into which the DNA is introduced is not particularly limited, as long as a gene of interest is expressed therein. The site may be on a plasmid or on a genome. Specific methods of introducing an expression cassette or a recombinant vector into a host may include, for example, a recombinant vector method and a genome editing method. Conditions for introducing an expression cassette or a recombinant vector into a host may be appropriately set according to the type of a host, etc. When the host is a bacterium, for example, a method using competent cells treated with calcium ions, an electroporation method, etc. are applied. When the host is a yeast, for example, an electroporation method, a spheroplast method, a lithium acetate method, etc. are applied. When the host is an animal cell, for example, an electroporation method, a calcium phosphate method, a lipofection method, etc. are applied. When the host is an insect cell, for example, a calcium phosphate method, a lipofection method, an electroporation method, etc. are applied. When the host is a plant cell, for example, an electroporation method, an Agrobacterium method, a particle gun method, a PEG method, etc. are applied. Whether or not the expression cassette or the recombinant vector has been incorporated into the host can be confirmed by a PCR method, a Southern hybridization method, a Northern hybridization method, etc.

In a case where whether or not the expression cassette or the recombinant vector has been incorporated into the host is confirmed by a PCR method, the genomic DNA, the expression cassette or the recombinant vector may be isolated and purified from the transformant, for example. When the host is a bacterium, for example, isolation and purification of the genomic DNA, the expression cassette or the recombinant vector may be performed by lysing the bacterium to obtain a lysate. When the bacteriolysis is performed, for example, a treatment is performed using a lytic enzyme such as lysozyme, and as necessary, protease, other enzymes, and surfactants such as sodium lauryl sulfate (SDS) are used in combination. In addition, when the bacteriolysis is performed, a physical disruption method such as freeze-thawing and French press treatment may be combined. The DNA can be separated from the lysate and purified, for example, by combining a deproteinization treatment involving a phenol treatment or a protease treatment, a ribonuclease treatment, an alcohol precipitation treatment, and a commercially available kit, with one another as appropriate.

The separated and purified DNA is used as a template, and primers specific to the DNA of the present invention are designed to perform PCR. The amplification product obtained by PCR is subjected to agarose gel electrophoresis, polyacrylamide gel electrophoresis, capillary electrophoresis, etc., and is then stained with ethidium bromide and SYBR Green solution, etc., and the amplification product is detected as a band, so that transformation can be confirmed. The DNA can be cleaved by a conventional method, for example, using a restriction enzyme treatment. For example, a type II restriction enzyme that acts on a specific nucleotide sequence is used as such a restriction enzyme.

Moreover, in case where whether or not the expression cassette or the recombinant vector has been incorporated into the host is confirmed by a PCR method, PCR is performed using primers that have been labeled with fluorescent dyes, etc. in advance, so that the amplification product can be detected. Furthermore, a method of binding the amplification product onto a solid phase such as a microplate and then confirming the amplification product by fluorescence and an enzyme reaction, etc. may also be adopted.

### (Method for Producing Oxygenase)

The present invention relates to a method for producing oxygenase, including a step of culturing the above-mentioned transformant.

The culture conditions for the step of culturing the transformant may be appropriately set in consideration of the nutritional and physiological properties of the aforementioned transformant, and liquid culture is preferably applied. In addition, upon performing industrial production, aeration and agitation culture is preferably applied. As a nutrient source for the medium, a source required for the growth of the transformant is used. The carbon source may be any assimilable carbon compound, and examples of the carbon source may include glucose, sucrose, lactose, maltose, molasses, and pyruvic acid. The nitrogen source may be any assimilable nitrogen compound, and examples of the nitrogen source may include peptone, a meat extract, a yeast extract, a casein hydrolysate, and an alkaline extract of soybean meal. In addition to the carbon and nitrogen sources, for example, salts such as phosphate, carbonate, sulfate, magnesium, calcium, potassium, iron, manganese and zinc, specific amino acids, and specific vitamins may also be used, as necessary.

The culture temperature is set, as appropriate, in a temperature range in which the above-mentioned transformant can grow and produces a predetermined oxygenase. For example, the culture temperature is preferably about 15° C. to 37° C. The culture may be completed at an appropriate time when the oxygenase reaches its maximum yield, and the culture time is usually about 12 to 48 hours.

After the step of culturing the transformant, the culture solution is subjected to a method such as centrifugation, and a culture supernatant and/or a cell mass are collected. The cell mass is treated by a mechanical method such as ultrasonication or French press, or with a lytic enzyme such as lysozyme, and as necessary, the cell mass is solubilized using an enzyme such as protease or a surfactant such as sodium lauryl sulfate (SDS), to obtain a water-soluble fraction containing an oxygenase of interest. In addition, the expressed oxygenase can also be secreted into the culture solution by selecting an appropriate expression cassette or an expression vector and a host.

The thus obtained water-soluble fraction containing oxygenase may be directly subjected to a purification treatment, but it is also possible that the oxygenase in the water-soluble fraction may be concentrated and may be then subjected to a purification treatment. The concentration can be carried out, for example, by vacuum concentration, membrane concentration, a salting-out treatment, or a fractional precipitation method using a hydrophilic organic solvent (e.g., methanol, ethanol, or acetone). Moreover, the purification treatment can be carried out, for example, by an appropriate combination of methods such as gel filtration, adsorption chromatography, ion exchange chromatography, and affinity chromatography. The purified oxygenase of interest may be pulverized by freeze drying, vacuum drying, spray drying, or the like, as necessary.

### (Method for Producing ω3-Epoxidized Fatty Acid)

The present invention relates to a method for producing ω3-epoxidized fatty acid, including a step of allowing the above-mentioned polypeptide or the above-mentioned enzyme agent for epoxidation of the position-ω3 to act on highly unsaturated fatty acid (ω3 fatty acid). By allowing the above-mentioned polypeptide (oxygenase) to act on ω3 fatty acid, fatty acid in which only the position-ω3 of ω3 fatty acid is epoxidized (ω3-epoxidized fatty acid) can be obtained. In other words, by treating ω3 fatty acid with the above-mentioned polypeptide (oxygenase), fatty acid in which only the position-ω3 of ω3 fatty acid is epoxidized (ω3-epoxidized fatty acid) can be obtained.

The ω3-epoxidized fatty acid obtained by the production method of the present invention is not particularly limited, and examples of the ω3-epoxidized fatty acid may include 19,20-epoxydocosapentaenoic acid (19,20-EpDPE), 19,20-epoxydocosatetraenoic acid (19,20-EpDTE), and 17,18-epoxyeicosatetraenoic acid (17,18-EpETE). Among them, the ω3-epoxidized fatty acid is preferably 17,18-epoxyeicosatetraenoic acid (17,18-EpETE).

The origin, properties, etc. of the ω3 fatty acid that serves the raw material in the production method of the ω3-epoxidized fatty acid of the present invention are not particularly limited. Specific examples of the ω3 fatty acid may include docosahexaenoic acid, docosapentaenoic acid, eicosapentaenoic acid, arachidonic acid, α-linolenic acid, stearidonic acid, 11,14,17-eicosatrienoic acid, tetracosapentaenoic acid, and tetracosahexaenoic acid, which are extracted from fish oil, linseed oil, perilla oil, fish oil and algae, and plants. As such ω3 fatty acid, these may be used alone or may also be used in combination of two or more types. Among these raw materials, the ω3 fatty acid is preferably at least one type selected from the group consisting of docosahexaenoic acid, docosapentaenoic acid, eicosapentaenoic acid, arachidonic acid, and α-linolenic acid, is more preferably at least one type selected from the group consisting of docosahexaenoic acid, docosapentaenoic acid, and eicosapentaenoic acid, and is further preferably eicosapentaenoic acid. The ω3 fatty acid may also be in the state of an oil or fat containing these fatty acids. The form of the ω3 fatty acid is not particularly limited, either, and it is preferably in the form of a liquid, a slurry, or a paste.

The amount of a polypeptide (oxygenase) used in the method for producing ω3-epoxidized fatty acid of the present invention is not particularly limited, and the oxygenase enzyme activity per mL of the reaction solution is preferably 0.0001 U or more. From the viewpoint of further enhancing the effects, the oxygenase enzyme activity per mL of the reaction solution is more preferably 0.0005 U or more, even more preferably 0.001 U or more, further preferably 0.005 U or more, and particularly preferably 0.01 U or more. The upper limit value of the oxygenase enzyme activity per mL of the reaction solution is not particularly limited, and for example, it is preferably 10 U or less, more preferably 5 U or less, even more preferably 1 U or less, further preferably 0.5 U or less, and still further preferably 0.1 U or less.

The enzyme activity of an oxygenase can be evaluated using the reduced amount of the coenzyme NADPH as an indicator. The amount of NADPH can be quantified by measuring the absorbance of the reaction solution at 360 nm using an absorption spectrometer. In addition, as a method of measuring the enzyme activity of an oxygenase, there is an analytical method of allowing an oxygenase to act on eicosapentaenoic acid (EPA) used as a substrate, and then quantifying the epoxidation product of the EPA using high performance liquid chromatography (HPLC). The consumption amount of the substrate and the amount of the generated product can be quantified from the area size calculated from the HPLC chart. When 3.3 mM EPA is used as the substrate and is treated at 35° C. for 1 hour, the amount of an enzyme that produces 1 mg of 17,18-EpETE per minute is defined as 1 unit (1 U).

In the method for producing ω3-epoxidized fatty acid of the present invention, other enzymes other than the above-described oxygenase may be used in combination. The enzyme(s) to be used in combination are preferably one or two or more enzymes selected from the group consisting of a NADPH-regenerating enzyme, an active oxygen-removing enzyme, and a hydrogen peroxide-removing enzyme. When the position-ω3 of ω3 fatty acid is epoxidized to produce ω3-epoxidized fatty acid, the reaction rate can be increased by using the above-described combined-use enzyme(s) in addition to the oxygenase, and the production rate of ω3-epoxidized fatty acid can be increased. With regard to the order of allowing individual enzymes to act on the substrate (i.e., the order of treatment with the oxygenase and the combined-use enzyme(s)), a simultaneous treatment is preferable.

Preferred aspects of the NADPH-regenerating enzyme, the active oxygen-removing enzyme and the hydrogen peroxide-removing enzyme are as those described in the above-mentioned section (Enzyme agent).

The amount of the NADPH-regenerating enzyme used in the method for producing ω3-epoxidized fatty acid of the present invention is not particularly limited, and the enzyme activity per mL of the reaction solution is preferably 0.1 U or more. From the viewpoint of further enhancing the effects, the NADPH-regenerating enzyme activity per mL of the reaction solution is more preferably 0.5 U or more, even more preferably 1.0 U or more, further preferably 3.5 U or more, and particularly preferably 7.0 U or more. The upper limit value of the NADPH-regenerating enzyme activity per mL of the reaction solution is not particularly limited, and for example, it is preferably 500 U or less, more preferably 100 U or less, even more preferably 50 U or less, further preferably 20 U or less, still further preferably 15 U or less, and particularly preferably 10 U or less.

The activity of the NADPH-regenerating enzyme is calculated by quantifying the amount of NADPH generated from the substrate NADP. NADPH can be quantified by measuring the absorbance of the reaction solution at 360 nm using an absorption spectrometer. The generation of 1 μM NADPH (mM) per minute is defined as 1 unit.

The amount of the active oxygen- or hydrogen peroxide-removing enzyme used in the method for producing ω3-epoxidized fatty acid of the present invention is not particularly limited, and the enzyme activity per mL of the reaction solution is preferably 0.1 U or more. From the viewpoint of further enhancing the effects, the activity of the active oxygen- or hydrogen peroxide-removing enzyme per mL of the reaction solution is more preferably 1 U or more, even more preferably 10 U or more, further preferably 25 U or more, and particularly preferably 45 U or more. The upper limit value of the activity of the active oxygen- or hydrogen peroxide-removing enzyme per mL of the reaction solution is not particularly limited, and for example, it is preferably 5000 U or less, more preferably 3000 U or less, even more preferably 1000 U or less, further preferably 100 U or less, still further preferably 75 U or less, and particularly preferably 60 U or less.

Among the active oxygen- or hydrogen peroxide-removing enzyme activities, catalase activity is calculated by decomposing 1.0 μmol of H₂O₂, and measuring the rate of decrease in the H₂O₂ concentration at an absorbance of 240 nm during the decomposition. The consumption of 1 μM H₂O₂ (mM) per minute is defined as 1 unit. Moreover, among the active oxygen- or hydrogen peroxide-removing enzyme activities, peroxidase activity is calculated by generating purpurogallin from pyrogallol and hydrogen peroxide and measuring the amount of color produced. The amount of the enzyme that produces 1 mg of purpurogallin from pyrogallol and hydrogen peroxide used as main substrates for 20 seconds is defined as 1 unit. Furthermore, among the active oxygen- or hydrogen peroxide-removing enzyme activities, superoxide dismutase activity is calculated by measuring the amount of superoxide radicals produced by xanthine oxidase using a tetrazolium salt. The amount of the enzyme required to dismutate 50% of superoxide radicals is defined as 1 unit.

The reaction time, the temperature, the pH of the reaction solution, etc., which are necessary for allowing an oxygenase(s) (one or two or more enzymes selected from the group consisting of a NADPH-regenerating enzyme, an active oxygen-removing enzyme, and a hydrogen peroxide-removing enzyme, as necessary) to act on ω3 fatty acid, are not particularly limited. The reaction temperature is, for example, preferably 20° C. to 50° C., more preferably 25° C. to 45° C., and further preferably 30° C. to 40° C. The pH of the reaction solution is, for example, preferably pH 5 to 10, more preferably pH 6 to 9.5, and further preferably pH 7 to 9. The reaction time is, for example, preferably 10 minutes to 48 hours, more preferably 1 to 24 hours, further preferably 2 to 12 hours, and particularly preferably 2 to 6 hours. Under the aforementioned reaction conditions, epoxidation of the position-ω3 of ω3 fatty acid can be more efficiently proceeded. These reaction conditions are selected, as appropriate, depending on epoxidized fatty acid of interest. Besides, the optimal reaction conditions may be determined through preliminary experiments.

After the step of allowing an oxygenase(s) and the like to act on ω3 fatty acid, a step of inactivating the oxygenase(s) (one or two or more enzymes selected from the group consisting of a NADPH-regenerating enzyme, an active oxygen-removing enzyme, and a hydrogen peroxide-removing enzyme, as necessary) may be provided.

By using the method for producing ω3-epoxidized fatty acid of the present invention, ω3-epoxidized fatty acid or a composition containing ω3-epoxidized fatty acid can be produced. That is to say, the present invention may relate to ω3-epoxidized fatty acid or a composition containing ω3-epoxidized fatty acid, which is obtained by the above-mentioned method for producing ω3-epoxidized fatty acid. In the ω3-epoxidized fatty acid-containing composition, preferably 80% by mass or more of, more preferably 90% by mass or more of, and further preferably 95% by mass or more of the fatty acid contained is ω3-epoxidized fatty acid.

### (Method for Epoxidizing ω3 Fatty Acid)

The present invention relates to a method for epoxidizing ω3 fatty acid, including a step of allowing the above-mentioned polypeptide or the above-mentioned enzyme agent for epoxidation of the position-ω3 to act on ω3 fatty acid. By allowing the above-mentioned polypeptide (oxygenase) to act on ω3 fatty acid, only the position-ω3 of the ω3 fatty acid can be epoxidized.

The above-mentioned combined-use enzyme can also be used in the method for epoxidizing ω3 fatty acid of the present invention. In addition, the reaction conditions in the method for epoxidizing ω3 fatty acid are the same as those in the step of allowing a polypeptide (oxygenase) to act on ω3 fatty acid in the above-mentioned method for producing ω3-epoxidized fatty acid.

### EXAMPLES

Hereinafter, the characteristics of the present invention will be more specifically described in the following examples. The materials, amounts used, proportions, treatment contents, treatment procedures, etc. shown in the following examples may be changed, as appropriate, without deviating from the gist of the present invention. Therefore, the scope of the present invention should not be interpreted limitedly by the specific examples shown below.

### (Test Method and Evaluation)

### 1. Search for Docking Structure and Design of Important Amino Acid Residues Involved in Enzyme-Substrate Reaction (1)

For the purpose of improving the epoxidation position specificity to the substrate EPA, a docking model of the enzyme-substrate complex structure was constructed, and MD simulation was performed, so as to predict important amino acid residues involved in the reaction of the position-ω3 of the substrate. Using the three-dimensional structure of P450-BM3 (P450 derived from Bacillus megaterium) (PDB number: 1bu7), and the substrate EPA, multiple patterns of MD simulation were performed for the purpose of simulating structural changes including the side chains or main chains of residues around the enzyme activity site. Various conditions were examined regarding the setting of a structural change promotion region that can take an energetically stable docking pose in which the substrate binds to the active site of the enzyme. As a result, conditions that make it easier for the EPA to move within the pocket were found. Further, in order to confirm the convergence of the calculation, calculations were performed twice with different initial conditions. As a result, the two independent results converged to the same amino acid residues. By comparing a structure in which the 17th and 18th positions of EPA contact the heme with a structure in which the 14th and 15th positions of EPA contact the heme, important amino acid residues involved in the reaction of the position-ω3 of the substrate were predicted. As a result, amino acid residues that had high scores in both of the two calculations were selected.

### 2. Activity Measurement of Mutant Enzyme (1)

Site-directed mutagenesis was performed on each of the seven amino acid residues that had high scores in the above MD calculations. Site-directed mutagenesis means that, regarding the selected amino acid residues, the original amino acid residues (wild type) are substituted with other amino acid residues (19 types) to prepare mutant enzymes. According to the method described below, 133 types of mutant enzymes with amino acid substitutions were prepared, and the enzymes were then reacted with the substrate (EPA). The products obtained after the reaction were analyzed by HPLC.

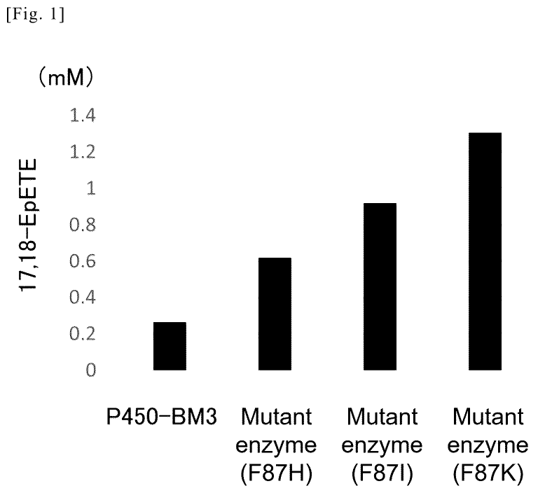

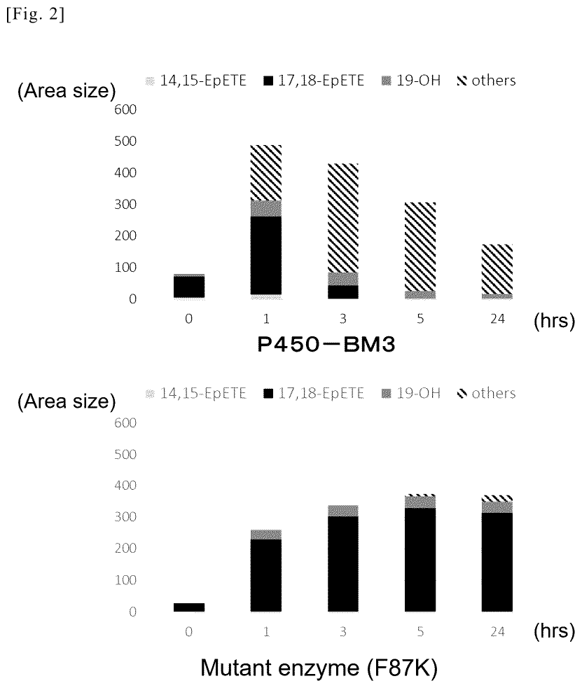

From the analysis of the HPLC chart, it was found that the composition of the product obtained after the reaction was significantly changed in mutant enzymes with a substitution of the phenylalanine at position 87. In particular, when the mutant enzymes (F87K, F87I, and F87H) were used, a reduction in by-products and accumulation of a product of interest, 17,18-EpETE (17,18-epoxyeicosatetraenoic acid), were detected (FIG. 1). Furthermore, as a result of the analysis of the enzyme-substrate reaction over time, it was demonstrated that the mutant enzyme (F87K) caused accumulation of the product of interest, 17,18-EpETE (FIG. 2, lower view).

The 17,18-EpETE production percentage and the production peak time of the mutant enzymes (F87K, F87I, and F87H) are shown in the table below.

TABLE 1

| | 17,18-EpETE | 17,18-EpETE |
|---|---|---|
| | production | production peak |
| | percentage | time |
| P450-BM3 | 45% | 1 hour |
| Mutant enzyme (F87H) | 71% | 4 hours |
| Mutant enzyme (F87I) | 65% | 2 hours |
| Mutant enzyme (F87K) | 83% | 4 hours |

### 3. Search for Docking Structure and Design of Important Amino Acid Residues Involved in Enzyme-Substrate Reaction (2)

For the purpose of further improving the reactivity, a second round of MD calculation and design was performed. The mutant enzyme (F87K) showed a decreased epoxidation reaction with EPA under conditions in which 17,18-EpETE was present, and it was considered that product inhibition might have occurred. Thus, a design was performed using the structure of P450-BM3 with the amino acid mutation F87K introduced, so that the product 17,18-EpETE would be more likely to separate from the enzyme.

Specifically, amino acid residues that do not affect the substrate EPA but destabilize the presence of the product 17,18-EpETE in the pocket were selected. As a result of two independent calculations, three types of amino acid residues were selected.

### 4. Activity Measurement of Mutant Enzyme (2)

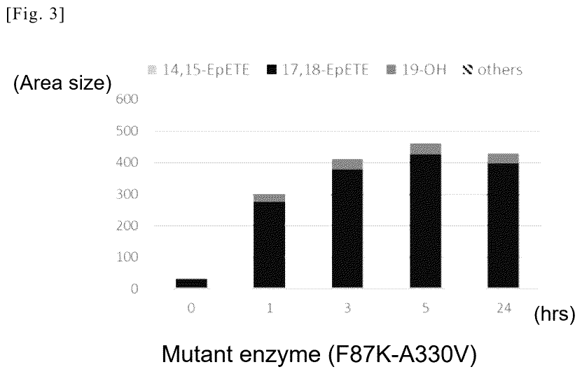

A double mutant enzyme further having an amino acid substitution in addition to F87K was prepared. Regarding the amino acid residues selected by the above-described MD calculation, 57 types of double mutant enzymes were prepared. The reaction was carried out under the conditions of <Condition 1> in <EPA epoxidation reaction and high performance liquid chromatography (HPLC) analysis> described below, and the products were then analyzed by HPLC. As a result, the amount of 17,18-EpETE produced was increased by multiple double mutant enzymes, compared to the mutant enzyme (F87K). In particular, the mutant enzyme (F87K-A330V) produced the highest amount of 17,18-EpETE. Further detailed analysis was performed to compare the enzyme-substrate reaction for a wild-type enzyme, the mutant enzyme (F87K), and the mutant enzyme (F87K-A330V). The position specificity of 17,18-EpETE was calculated, and as a result, the maximum production percentage of 17,18-EpETE from EPA was 44% in the case of the wild-type enzyme, 89% in the case of the mutant enzyme (F87K), and 93% in the case of the mutant enzyme (F87K-A330V) (FIG. 2 and FIG. 3). These results suggested the possibility of industrial production of 17,18-EpETE, which had not been possible with the wild-type enzyme.

### 5. Search for Docking Structure and Design of Important Amino Acid Residues Involved in Enzyme-Substrate Reaction (3)

For the purpose of further improving the position specificity of the mutant enzyme (F87K-A330V) and eliminating product inhibition, a third round of MD calculation was performed. The three-dimensional structure of P450-BM3 with the amino acid mutation F87K-A330V introduced was generated by modeling. The MD calculation was performed on that structure, and a mutant enzyme that destabilizes the product-inhibiting structure while maintaining the product 17,18-EpETE was predicted, and was designed. A structure in which a product (epoxidized substrate) does not stably bind was predicted, and substituted amino acids were designed. As a result, three amino acid residues were selected. In the previous two designs, mutations were introduced into the region close to the heme, which is the active center, whereas in this design, mutations were introduced into amino acid residues located far from the active center.

### 6. Activity Measurement of Mutant Enzyme (3)

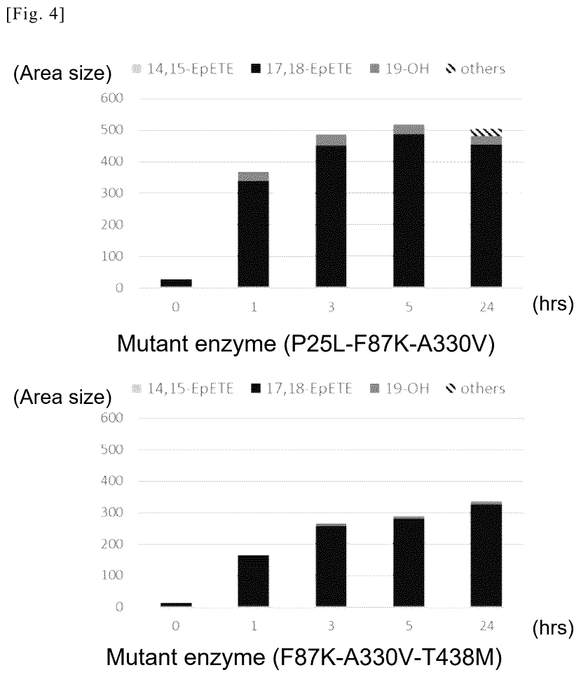

As before, site-specific mutagenesis was performed on the selected amino acid residues. Fifty-seven types of triple mutant enzymes further having an amino acid substitution, in addition to F87K and A330V, were prepared by PCR. These triple mutant enzymes were reacted with the substrate EPA, and the resulting products were analyzed by HPLC. As a result, it was found that the mutant enzyme (P25L-F87K-A330V) and mutant enzyme (F87K-A330V-T438M) showed improved epoxidation position specificity and a high production percentage of 17,18-EpETE from EPA (FIG. 4). In particular, the mutant enzyme (F87K-A330V-T438M) showed improved epoxidation position specificity and a production percentage of 17,18-EpETE from EPA of 96% (FIG. 4, lower view). Furthermore, in the case of the mutant enzyme (P25L-F87K-A330V), the amount of EPA consumed was improved, and elimination of product inhibition was observed.

7. Combined Use with NADPH-Regenerating Enzyme/Hydrogen Peroxide-Removing Enzyme

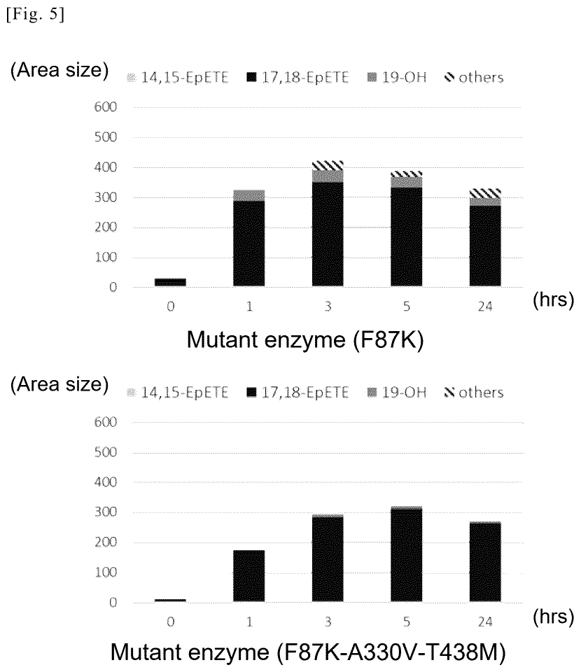

In order to evaluate the effect of the combined use with other enzymes, the reaction was carried out under the conditions of <Condition 2: Combined use with NADPH-regenerating enzyme/hydrogen peroxide-removing enzyme> described below. In addition, a reaction solution containing glucose, which is essential for the catalytic action of glucose dehydrogenase, was prepared. Using a frozen Escherichia coli cell mass expressing the mutant enzyme (F87K) and a frozen Escherichia coli cell mass expressing the mutant enzyme (F87K-A330V-T438M), the production percentage and production amount of 17,18-EpETE were evaluated. As a result, it was found that the production percentage of 17,18-EpETE by the mutant enzyme (F87K) was slightly decreased to 85%, compared to the case of no combined use (FIG. 5, upper view). However, the peak time of production amount was 3 hours, and thus, it was found that the enzymatic reaction proceeded in a shorter time than that of no combined use (FIG. 5, upper view). Furthermore, the production percentage of 17,18-EpETE by the triple mutant enzyme (F87K-A330V-T438M) was 97%, compared to the case of no combined use, and thus, the production percentage was further improved (FIG. 5, lower view). The peak time of production amount was 5 hours, and thus, it was found that the enzyme reaction proceeded in a shorter time than that of no combined use (FIG. 5, lower view). From the above results, the combined use effect was observed to improve the substrate-converting speed, when compared with the same reaction time.

### (Method for Producing Mutant Enzyme)

### (1) Construction of P450-BM3 Expression Vector

The CYP102A1 gene amplified by PCR from the genome of Bacillus megaterium was inserted into the multicloning site, BamHI-EcoRI, of the expression plasmid pET28a (Merck & Co.). Specifically, in the PCR reaction, primers, to which a restriction enzyme site had been added (Table 2), and Prime STAR MAX DNA Polymerase (Takara Bio, Inc.) were used. The PCR-amplified gene sequence was ligated to the vector, using DNA Ligation Kit <Mighty Mix> (Takara Bio, Inc.). The DNA sequence of the gene was confirmed by Sanger sequencing. The DNA sequence of the gene was as shown in SEQ ID No: 1.

TABLE 2

| Primer Name | Sequence (5′→3′) |
|---|---|
| P450-BM3-FW | gggaccatgggcagcagccatcatca |
| P450-BM3-RV | ggggaattcttacccagcccacacgtc |

### (2) Preparation of Recombinant P450-BM3

The constructed P450-BM3 expression vector (1 μL) was mixed with 2 μL of E. coli BL21 (DE3) (Nippon Gene Co., Ltd.) for transformation. After 20 minutes of recovery culture at 37° C., the transformation solution was applied onto an LB agar medium plate (manufactured by Invitrogen Co., Ltd.) supplemented with kanamycin (final concentration 50 μg/mL) and was then cultured at 37° C. for 18 hours. The grown colonies were inoculated into an LB medium (manufactured by Invitrogen Co., Ltd.) supplemented with kanamycin (final concentration 50 μg/mL). The culture was carried out at 37° C. for 18 hours to prepare a preculture solution. The preculture solution was inoculated into 1 mL of a TB medium (manufactured by Invitrogen Co. Ltd.) supplemented with kanamycin (final concentration 50 μg/mL). The culture was carried out at 37° C. for 48 hours (IPTG (final concentration: 0.1 mM) was added at a time point of 24 hours after initiation of the culture), and thereafter, the cell mass was recovered by centrifugation.

### (3) Construction of Expression Vector for Mutants P450-BM3 (F87K, F87H, and F87I)

Mutation-introducing PCR primers (Table 3) were designed, and PCR was carried out under the following conditions, so that mutations were introduced into the CYP102A1 gene.

TABLE 3

| Primer Name | Sequence (5′→3′) |
|---|---|
| P450BM3-F87K-FW | gcaggagacgggttaaagacaagctggacgcatgaaaaa |
| | aat |
| P450BM3-F87H-FW | gcaggagacgggttacatacaagctggacgcatgaaaaa |
| | aat |
| P450BM3-F87I-FW | gcaggagacgggttaatcacaagctggacgcatgaaaaa |
| | aat |
| P450BM3-F87-RV | taacccgtctcctgcaaaatcacg |

### <Template Plasmid>

P450-BM3 expression vector: about 50 ng/μL
   <Composition of PCR reaction> (Total amount: 25 μL)
Prime STAR MAX DNA Polymerase (Takara Bio, Inc.): 12.5 μL
Template plasmid: 0.5 L
Each primer (the combination of primers are as shown in Table 4): 0.5 μL×2
Ultrapure water (Milli Q water): 11 μL

TABLE 4

| Mutation Point | Primer 1 | Primer 2 |
|---|---|---|
| F87K | P450BM3-F87K-FW | P450BM3-F87-RV |
| F87H | P450BM3-F87H-FW | P450BM3-F87-RV |
| F87I | P450BM3-F87I-FW | P450BM3-F87-RV |

### <PCR Conditions>

At 98° C. for 30 seconds,
A reaction consisting of 98° C.-10 seconds and 68° C.-8 minutes was repeated by 10 cycles,
At 72° C. for 10 minutes,
Left at 4° C.

### <Restriction Enzyme Treatment and Purification>

0.5 L of the restriction enzyme DpnI (manufactured by Takara Bio, Inc.) was added to 20 μL of the PCR reaction solution, and the obtained mixture was then reacted at 37° C. for 3 hours or more. DNA was purified using the DNA purification kit NucleoSpin Gel and PCR Clean-up (manufactured by Takara Bio, Inc.).

### <In-Fusion Reaction>

The reaction solution with the following composition was prepared (total volume 10 μL), and was then heated at 50° C. for 15 minutes in a thermal cycler.
Purified PCR solution: 2 to 5 μL (corresponding to 50 to 200 ng)
Milli Q water: 6 to 3 μL
5× In-Fusion HD Enzyme Premix: 2 μL

5× In-Fusion HD Enzyme Premix is a reagent included in the In-Fusion® HD Cloning Kit (Takara Bio, Inc.).

<Escherichia coli Transformation>

2 μL of the in-fusion reaction solution was mixed into 18 μL of E. coli BL21 (DE3) (NIPPON GENE CO., LTD.) to perform transformation. After 20 minutes of recovery culture at 37° C., the transformation solution was applied onto an LB agar medium plate (manufactured by Invitrogen Co., Ltd.) supplemented with kanamycin (final concentration: 50 μg/mL), and the obtained mixture was then cultured at 37° C. for 18 hours.

### <Plasmid Purification and Sequence Confirmation>

The grown colonies were inoculated into an LB medium (manufactured by Invitrogen Co., Ltd.) supplemented with kanamycin (final concentration: 50 μg/mL). The cells were cultured at 37° C. for 18 hours to obtain a cell mass culture solution. The cell mass was recovered by centrifugation, and each plasmid was then purified using the plasmid miniprep kit NucleoSpin Plasmid EasyPure (Takara Bio, Inc.). The DNA sequences of the genes were confirmed by Sanger sequencing. The DNA sequences of the mutant enzymes (F87K, F87I, and F87H) genes were as shown in SEQ ID Nos: 3, 5, and 7, respectively.

### (4) Preparation of Mutation-Type Recombinants P450-BM3 (F87K, F87H, and F87I)

The constructed mutation-type P450-BM3 expression vector (1 μL) was mixed with 2 μL of E. coli BL21 (DE3) (Nippon Gene Co., Ltd.) for transformation. After 20 minutes of recovery culture at 37° C., the transformation solution was applied onto an LB agar medium plate (manufactured by Invitrogen Co., Ltd.) supplemented with kanamycin (final concentration 50 μg/mL) and was then cultured at 37° C. for 18 hours. The grown colonies were inoculated into an LB medium (manufactured by Invitrogen Co., Ltd.) supplemented with kanamycin (final concentration 50 μg/mL). The culture was carried out at 37° C. for 18 hours to prepare a preculture solution. The preculture solution was inoculated into 1 mL of a TB medium (manufactured by Invitrogen Co. Ltd.) supplemented with kanamycin (final concentration 50 μg/mL). The culture was carried out at 37° C. for 48 hours (IPTG (final concentration: 0.1 mM) was added at a time point of 24 hours after initiation of the culture), and thereafter, the cell mass was recovered by centrifugation. The amino acid sequences of the obtained mutation-type recombinants P450-BM3 (F87K, F87H, and F87I) are as shown in SEQ ID Nos: 4, 6, and 8, respectively.

### (5) Preparation of Double Mutation-Type Recombinant P450-BM3 (F87K-A330V)

A mutation-type P450-BM3 (F87K-A330V) expression vector, in which double mutations were introduced into the CYP102A1 gene, was obtained by performing PCR, etc., under the same conditions as those in the above-mentioned method, with the exceptions that mutation-introducing PCR primers (Table 5) were designed, and that a mutation-type P450-BM3 (F87K) expression vector was used as a template plasmid. The DNA sequence of the gene was as shown in SEQ ID No: 9. A cell mass expressing the mutation-type recombinant P450-BM3 (F87K-A330V) was recovered by the same method as the above-mentioned method, with the exception that the mutation-type P450-BM3 (F87K-A330V) expression vector was used instead of the mutation-type P450-BM3 expression vector. The amino acid sequence of the obtained mutation-type recombinant P450-BM3 was as shown in SEQ ID No: 10.

TABLE 5

| Primer Name | Sequence (5′→3′) |
|---|---|
| P450BM3-A330V-FW | cgcttatggccaactgctcctgttttttccctatatgcaaaaga |
| P450BM3-A330-RV | agttggccataagcgcagcg |

### (6) Preparation of Triple Mutation-Type Recombinant P450-BM3 (P25L-F87K-A330V)

A mutation-type P450-BM3 (P25L-F87K-A330V) expression vector, in which triple mutations were introduced into the CYP102A1 gene, was obtained by performing PCR, etc., under the same conditions as those in the above-mentioned method, with the exceptions that mutation-introducing PCR primers (Table 6) were designed, and that a double mutation-type P450-BM3 (F87K-A330V) expression vector was used as a template plasmid. The DNA sequence of the gene was as shown in SEQ ID No: 11. A cell mass expressing the mutation-type recombinant P450-BM3 (P25L-F87K-A330V) was recovered by the same method as the above-mentioned method, with the exception that the mutation-type P450-BM3 (P25L-F87K-A330V) expression vector was used instead of the mutation-type P450-BM3 expression vector. The amino acid sequence of the obtained mutation-type recombinant P450-BM3 was as shown in SEQ ID No: 12.

TABLE 6

| Primer Name | Sequence (5′→3′) |
|---|---|
| P450BM3-P25L-FW | ttaaacacagataaacttgttcaagctttgatgaaaattgc |
| P450BM3-P25-RV | tttatctgtgtttaataacggtaaatt |

### (7) Preparation of Mutation-Type Recombinant P450-BM3 (F87K-A330V-T438M)

A mutation-type P450-BM3 (F87K-A330V-T438M) expression vector, in which triple mutations were introduced into the CYP102A1 gene, was obtained by performing PCR, etc., under the same conditions as those in the above-mentioned method, with the exceptions that mutation-introducing PCR primers (Table 7) were designed, and that a mutation-type P450-BM3 (F87K-A330V) expression vector was used as a template plasmid. The DNA sequence of the gene was as shown in SEQ ID No: 13. A cell mass expressing the mutation-type recombinant P450-BM3 (F87K-A330V-T438M) was recovered by the same method as the above-mentioned method, with the exception that the mutation-type P450-BM3 (F87K-A330V-T438M) expression vector was used instead of the mutation-type P450-BM3 expression vector. The amino acid sequence of the obtained mutation-type recombinant P450-BM3 was as shown in SEQ ID No: 14.

TABLE 7

| Primer Name | Sequence (5′→3′) |
|---|---|
| P450BM3-T438M-FW | gatattaaagaaactttaatgttaaaacctgaaggctttgtggta |
| P450BM3-T438-RV | agtttctttaatatccagctcgtagtt |

### (Activity Measurement Method)

### <EPA Epoxidation Reaction and High Performance Liquid Chromatography (HPLC) Analysis>

### <Condition 1>

A 0.1 M Tris-HCl buffer (pH 8.0) containing 3.3 mM substrate EPA (dissolved in ethanol; manufactured by Combi-Blocks) and 3 mM NADPH (manufactured by Oriental Yeast Co., Ltd.) was added to a frozen Escherichia coli cell mass expressing P450-BM3 or a frozen Escherichia coli cell mass expressing mutation-type P450-BM3, and the obtained mixture was then mixed well. The reaction was carried out at 35° C., while mixing it with a maximizer. The reaction solution was sampled, when 0, 1, 3, 5, and 24 hours have passed. The sampled reaction solution was 40 times diluted with 100% ethanol (manufactured by FUJIFILM Wako Pure Chemical Corporation). The diluted sample was transferred to a vial, and HPLC analysis was performed.

<Condition 2: Combined Use with NADPH-Regenerating Enzyme/Hydrogen Peroxide-Removing Enzyme>

A 0.1 M Tris-HCl buffer (pH 8.0) containing 3.3 mM substrate EPA (dissolved in ethanol; manufactured by Combi-Blocks), 0.16 M glucose (manufactured by FUJIFILM Wako Pure Chemical Corporation), 7.7 U/mL glucose dehydrogenase (NAD type) (manufactured by Amano Enzyme Inc.), 50 U/ml catalase (manufactured by FUJIFILM Wako Pure Chemical Corporation) and 3 mM NADPH (manufactured by Oriental Yeast Co., Ltd.) was added to a frozen Escherichia coli cell mass expressing P450-BM3 or a frozen Escherichia coli cell mass expressing mutation-type P450-BM3, and the obtained mixture was then mixed well. The reaction was carried out at 35° C., while mixing it with a maximizer. The reaction solution was sampled, when 0, 1, 3, 5, and 24 hours have passed. The sampled reaction solution was 400 times diluted with 100% ethanol (manufactured by FUJIFILM Wako Pure Chemical Corporation). The diluted sample was transferred to a vial, and HPLC analysis was performed.

HPLC analysis was carried out on the epoxidized metabolites produced from the substrate EPA. Conditions for the HPLC analysis were as follows.
Analysis column: COSMOSIL 5C18 AR-II, 250×4.6 mm, 5.0 μm (Nacalai Tesque, Inc.)
Detector: UV 205 nm
Flow rate: 1.2 ml/min
Column temperature: 35° C.

Buffer and program were as follows.

TABLE 8

Solvent Ratio (% by weight)

| | Milli Q water | Acetonitrile |
|---|---|---|
| Time (min) | (0.02% acetic acid) | (0.02% acetic acid) |
| 0 | 40 | 60 |
| 3 | 40 | 60 |
| 10 | 0 | 100 |
| 12 | 0 | 100 |
| 12.01 | 40 | 60 |
| 17 | 40 | 60 |

The peak areas were quantified to determine the amounts of EPA, 17,18-EpETE (17,18-epoxyeicosatetraenoic acid), 14,15-EpETE (14,15-epoxyeicosatetraenoic acid), and 19-HEPE (19-hydroxyl eicosapentaenoic acid). The peak positions of 17,18-EpETE and 14,15-EpETE were determined using their respective standard preparations (manufactured by Cayman Chemical). Regarding 19-HEPE, the peak was fractionated after the HPLC analysis, and the structure thereof was specified by NMR. It is to be noted that, in FIGS. 2 to 4, “others” include secondary metabolites in which both the positions-ω3 and -ω6 are epoxidized, and unidentified metabolites (those in which multiple positions of the substrate were epoxidized, those in which double bonds other than the positions-ω3 and -ω6 were epoxidized, those in which hydroxyl groups are added positions that are not double bonds other than the 19-OH, etc.).

It is to be noted that the 17,18-EpETE production percentage was calculated using the following formula:

$$17 , 18 - EpETE production amount ( \% ) = area size of 17 , \\ 18 - EpETE / ( area size of 17 , 18 - EpETE + area size of 14 , \\ 15 - EpETE + area size of 19 - OH + area sizes of others ) \times 100$$

### <DHA Epoxidation Reaction and High Performance Liquid Chromatography (HPLC) Analysis>

### <Condition 3>

A 0.1 M Tris-HCl buffer (pH 8.0) containing 3.3 mM substrate DHA (dissolved in ethanol; manufactured by MedChemExpress) and 3 mM NADPH (manufactured by Oriental Yeast Co., Ltd.) was added to a frozen Escherichia coli cell mass expressing P450-BM3 or a frozen Escherichia coli cell mass expressing mutation-type P450-BM3, and the obtained mixture was then mixed well. The reaction was carried out at 35° C., while mixing it with a maximizer. The reaction solution was sampled, when 0, 1, 3, 5, and 24 hours have passed. The sampled reaction solution was 40 times diluted with 100% ethanol (manufactured by FUJIFILM Wako Pure Chemical Corporation). The diluted sample was transferred to a vial, and HPLC analysis was performed. Besides, the HPLC analysis was carried out under the same conditions as those described above.

The peak areas were quantified to determine the amounts of DHA, 19,20-EpDPE (19,20-epoxydocosapentaenoic acid) and 16,17-EpDPE (16,17-epoxydocosapentaenoic acid). The peak positions of 19,20-EpDPE and 16,17-EpDPE were determined using their respective standard preparations (manufactured by Cayman Chemical). Regarding “others,” the area sizes of unidentified peaks were summed.

It is to be noted that the 19,20-EpDPE production percentage was calculated using the following formula:

$$19 , 20 - EpDPE production amount ( \% ) = area size of 19 , \\ 20 - EpDPE / ( area size of 19 , 20 - EpDPE + area size of 16 , \\ 17 - EpDPE + area sizes of others ) \times 100$$

By reacting the 5 types of mutant enzymes prepared above with the substrate DHA, 19,20-EpDPE (19,20-epoxydocosapentaenoic acid) and 16,17-EpDPE (16,17-epoxydocosapentaenoic acid) were produced. 19,20-EpDPE (19,20-epoxydocosapentaenoic acid), which is produced by epoxidizing the position-ω3 of DHA, has physiological effects similar to those of 17,18-EpETE and is a useful product.

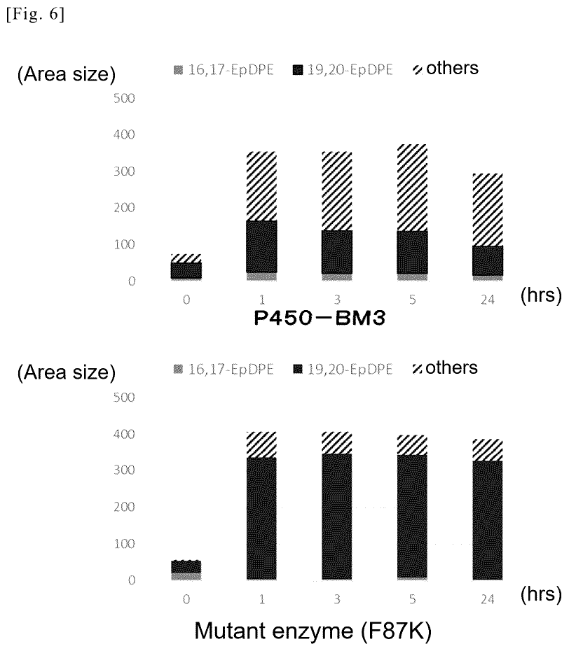

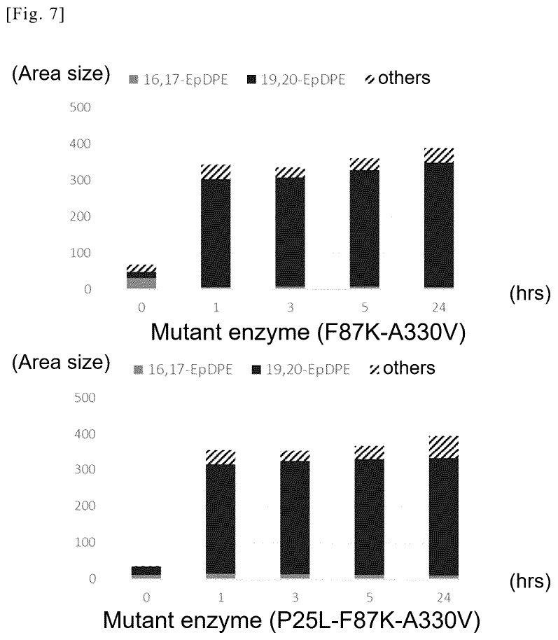

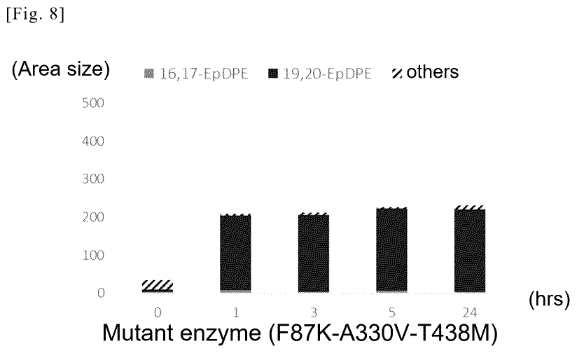

Compared to the wild-type enzyme (P450-BM3), by conversion using the mutant enzyme (F87K), the mutant enzyme (F87K-A330V), the mutant enzyme (P25L-F87K-A330V), and the mutant enzyme (F87K-A330V-T438M), an increase in the production amount of 19,20-EpDPE and accumulation thereof were confirmed (FIGS. 6 to 8).

In addition, when the position specificity of 19,20-EpDPE was calculated, the maximum value of the production percentage of 19,20-EpDPE from DHA was 40% in the wild type, whereas it was 84% in the mutant enzyme (F87K), 90% in the mutant enzyme (F87K-A330V), 88% in the mutant enzyme (P25L-F87K-A330V), and 96% in the mutant enzyme (F87K-A330V-T438M) (FIGS. 6 to 8). These results suggested the possibility of industrial production of 19,20-EpDPE, which had not been possible with the wild-type enzyme.

### <DPA Epoxidation Reaction and High Performance Liquid Chromatography (HPLC) Analysis>

### <Condition 4>

A 0.1 M Tris-HCl buffer (pH 8.0) containing 3.3 mM substrate DPA (dissolved in ethanol; manufactured by Cayman Chemical) and 3 mM NADPH (manufactured by Oriental Yeast Co., Ltd.) was added to a frozen Escherichia coli cell mass expressing P450-BM3 or a frozen Escherichia coli cell mass expressing mutation-type P450-BM3, and the obtained mixture was then mixed well. The reaction was carried out at 35° C., while mixing it with a maximizer. The reaction solution was sampled, when 0, 1, 3, 5, and 24 hours have passed. The sampled reaction solution was 40 times diluted with 100% ethanol (manufactured by FUJIFILM Wako Pure Chemical Corporation). The diluted sample was transferred to a vial, and HPLC analysis was performed. Besides, the HPLC analysis was carried out under the same conditions as those described above.

The peak areas were quantified to determine the amounts of DPA and 19,20-EpDTE (19,20-epoxydocosatetraenoic acid). The peak position of 19,20-EpDTE was determined using the standard preparation (manufactured by Cayman Chemical). Regarding “others,” the area sizes of unidentified peaks were summed.

It is to be noted that the 19,20-EpDPE production percentage was calculated using the following formula:

$$19 , 20 - EpDPE production amount ( \% ) = area size of 19 , \\ 20 - EpDPE / ( area size of 19 , 20 - EpDPE + area sizes of others ) \times 100$$

By reacting the 5 types of mutant enzymes prepared above with the substrate DPA, 19,20-EpDTE (19,20-epoxydocosatetraenoic acid) was produced. 19,20-EpDTE (19,20-epoxydocosatetraenoic acid), which is produced by epoxidizing the position-ω3 of DPA, has physiological effects similar to those of 17,18-EpETE and is a useful product.

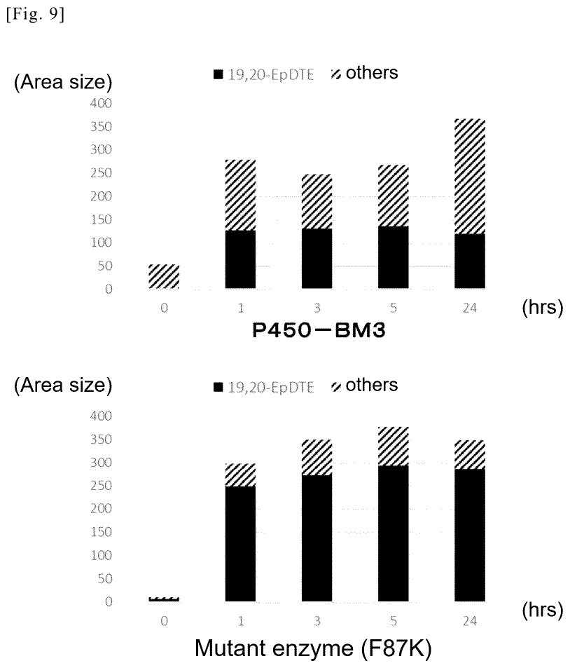

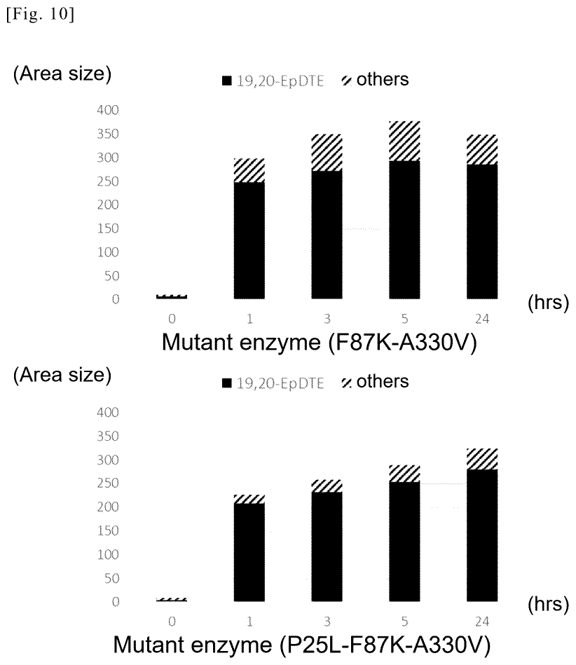

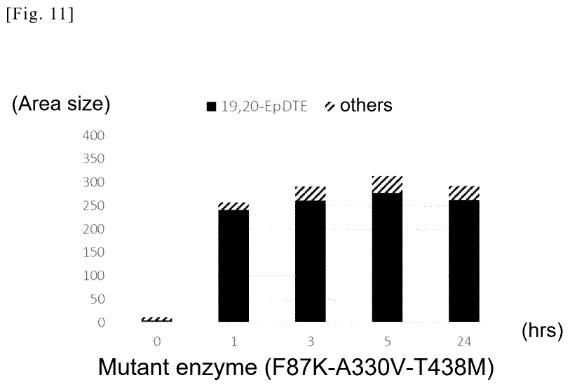

Compared to the wild-type enzyme (P450-BM3), by conversion using the mutant enzyme (F87K), the mutant enzyme (F87K-A330V), the mutant enzyme (P25L-F87K-A330V), and the mutant enzyme (F87K-A330V-T438M), an increase in the production amount of 19,20-EpDTE and accumulation thereof were confirmed (FIGS. 9 to 11).

In addition, when the position specificity of 19,20-EpDTE was calculated, the maximum value of the production percentage of 19,20-EpDTE from DPA was 52% in the wild type, whereas it was 78% in the mutant enzyme (F87K), 89% in the mutant enzyme (F87K-A330V), 89% in the mutant enzyme (P25L-F87K-A330V), and 94% in the mutant enzyme (F87K-A330V-T438M) (FIGS. 9 to 11). These results suggested the possibility of industrial production of 19,20-EpDTE, which had not been possible with the wild-type enzyme.

### INDUSTRIAL APPLICABILITY

According to the present invention, there can be obtained a modified oxygenase that specifically epoxidizes only the position-ω3 of highly unsaturated fatty acid. In addition, according to the present invention, it becomes possible to industrially produce ω3-epoxidized fatty acid, in which only the position-ω3 is specifically epoxidized.

### SEQ ID No: 1

### gcg gat gaa tta gga gaa atc ttt aaa ttc gag gcg cot ggt cgt gta Ala Asp Glu Leu Gly Glu Ile Phe Lys Phe Glu Ala Pro Gly Arg Val

1. A polypeptide described in any of the following (1) to (3):
(1) a polypeptide consisting of an amino acid sequence containing one or two or more amino acid substitutions selected from the group consisting of F87K/I/H, A330V, P25L and T438M, in the amino acid sequence as set forth in SEQ ID No: 2;
(2) a polypeptide containing a substitution, addition, insertion or deletion of one or several amino acid residues in the amino acid sequence described in the above (1), having a catalytic activity of epoxidizing the position-ω3 of ω3 fatty acid, and retaining one or two or more amino acid substitutions selected from the group consisting of F87K/I/H, A330V, P25L and T438M; and
(3) a polypeptide having a sequence identity of 90% or more to the amino acid sequence described in the above (1), having a catalytic activity of epoxidizing the position-ω3 of ω3 fatty acid, and retaining one or two or more amino acid substitutions selected from the group consisting of F87K/I/H, A330V, P25L and T438M,
(provided that F87K/I/H indicates that F, the amino acid residue at position 87 in SEQ ID No: 2, is substituted with K, I or H; A330V indicates that A, the amino acid residue at position 330 in SEQ ID No: 2, is substituted with V; P25L indicates that P, the amino acid residue at position 25 in SEQ ID No: 2, is substituted with L; and T438M indicates that T, the amino acid residue at position 438 in SEQ ID No: 2, is substituted with M).

2. The polypeptide according to claim 1, in which the sequence identity in the above (3) is 95% or more.

3. The polypeptide according to claim 1, which is an oxygenase.

4. An enzyme agent for epoxidation of the position-ω3 of ω3 fatty acid, containing the polypeptide according to claim 1.

5. The enzyme agent for epoxidation of the position-ω3 according to claim 4, further containing one or two or more enzymes selected from the group consisting of a NADPH-regenerating enzyme, an active oxygen-removing enzyme, and a hydrogen peroxide-removing enzyme.

6. DNA described in any of the following (1) to (3):
(1) DNA encoding the polypeptide according to claim 1;
(2) DNA consisting of the nucleotide sequence as set forth in any of SEQ ID Nos: 3, 5, 7 and 9; and
(3) DNA containing a sequence equivalent to the nucleotide sequence as set forth in any of SEQ ID Nos: 3, 5, 7 and 9, and encoding a polypeptide having a catalytic activity of epoxidizing the position-ω3 of ω3 fatty acid.

7. An expression cassette, containing the DNA according to claim 6.

8. A recombinant vector, containing the DNA according to claim 6.

9. A transformant, having the DNA according to claim 6.

10. A method for producing an oxygenase, including
culturing the transformant according to claim 9.

11. A method for producing ω3-epoxidized fatty acid, including
allowing the polypeptide according to claim 1 to act on ω3 fatty acid.

12. A method for producing ω3-epoxidized fatty acid, including
allowing the enzyme agent for epoxidation of the position-ω3 according to claim 4 to act on ω3 fatty acid.

13. A method for epoxidizing ω3 fatty acid, including
allowing the polypeptide according to claim 1 to act on ω3 fatty acid.

14. A method for epoxidizing ω3 fatty acid, including
allowing the enzyme agent for epoxidation of the position-ω3 according to claim 4 to act on ω3 fatty acid.

15. A composition, containing the ω3-epoxidized fatty acid, which is obtained by the production method according to claim 11.

16. A composition, containing, the ω3-epoxidized fatty acid, which is obtained by the production method according to claim 12.

17. A transformant, having the expression cassette according to claim 7.

18. A transformant, having the recombinant vector according to claim 8.


SEQUENCE LISTING (26 sequences)

SEQ ID NO: 1 - DNA, 3144 bp, Bacillus megaterium
  source 1..3144: mol_type = genomic DNA
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttatttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctggg

SEQ ID NO: 2 - protein, 1048 aa, Bacillus megaterium
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 3 - DNA, 3144 bp, synthetic construct
  source 1..3144: mol_type = other DNA
  CDS 1..3144
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttaaagacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctggg

SEQ ID NO: 4 - protein, 1048 aa, synthetic construct
TIKEMPQPKTFGELKNLPLLNTDKPVQALMKIADELGEIFKFEAPGRVTRYLSSQRLIKEACDESRFDKNLSQALKFVRDFAGDGLKTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 5 - DNA, 3144 bp, synthetic construct
  source 1..3144: mol_type = other DNA
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttacatacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctggg

SEQ ID NO: 6 - protein, 1048 aa, synthetic construct
TIKEMPQPKTFGELKNLPLLNTDKPVQALMKIADELGEIFKFEAPGRVTRYLSSQRLIKEACDESRFDKNLSQALKFVRDFAGDGLHTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 7 - DNA, 3144 bp, synthetic construct
  source 1..3144: mol_type = other DNA
  CDS 1..3144
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttaatcacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctggg

SEQ ID NO: 8 - protein, 1048 aa, synthetic construct
TIKEMPQPKTFGELKNLPLLNTDKPVQALMKIADELGEIFKFEAPGRVTRYLSSQRLIKEACDESRFDKNLSQALKFVRDFAGDGLITSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 9 - DNA, 3144 bp, synthetic construct
  source 1..3144: mol_type = other DNA
  CDS 1..3144
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttaaagacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgttttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctggg

SEQ ID NO: 10 - protein, 1048 aa, synthetic construct
TIKEMPQPKTFGELKNLPLLNTDKPVQALMKIADELGEIFKFEAPGRVTRYLSSQRLIKEACDESRFDKNLSQALKFVRDFAGDGLKTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPVFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 11 - DNA, 3144 bp, synthetic construct
  source 1..3144: mol_type = other DNA
  CDS 1..3144
acaattaaagaaatgcctcagccaaaaacgtttggagagcttaaaaatttaccgttattaaacacagataaacttgttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttaaagacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgttttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctggg

SEQ ID NO: 12 - protein, 1048 aa, synthetic construct
TIKEMPQPKTFGELKNLPLLNTDKLVQALMKIADELGEIFKFEAPGRVTRYLSSQRLIKEACDESRFDKNLSQALKFVRDFAGDGLKTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPVFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 13 - DNA, 3144 bp, synthetic construct
  source 1..3144: mol_type = other DNA
  CDS 1..3144
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttaaagacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgttttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaatgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctggg

SEQ ID NO: 14 - protein, 1048 aa, synthetic construct
TIKEMPQPKTFGELKNLPLLNTDKPVQALMKIADELGEIFKFEAPGRVTRYLSSQRLIKEACDESRFDKNLSQALKFVRDFAGDGLKTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPVFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLMLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 15 - DNA, 26 bp, synthetic construct
  misc_feature 1..26: note = Description of Artificial Sequence: primer
  source 1..26: mol_type = other DNA
gggaccatgggcagcagccatcatca

SEQ ID NO: 16 - DNA, 27 bp, synthetic construct
  misc_feature 1..27: note = Description of Artificial Sequence: primer
  source 1..27: mol_type = other DNA
ggggaattcttacccagcccacacgtc

SEQ ID NO: 17 - DNA, 42 bp, synthetic construct
  misc_feature 1..42: note = Description of Artificial Sequence: primer
  source 1..42: mol_type = other DNA
gcaggagacgggttaaagacaagctggacgcatgaaaaaaat

SEQ ID NO: 18 - DNA, 42 bp, synthetic construct
  misc_feature 1..42: note = Description of Artificial Sequence: primer
  source 1..42: mol_type = other DNA
gcaggagacgggttacatacaagctggacgcatgaaaaaaat

SEQ ID NO: 19 - DNA, 42 bp, synthetic construct
  misc_feature 1..42: note = Description of Artificial Sequence: primer
  source 1..42: mol_type = other DNA
gcaggagacgggttaatcacaagctggacgcatgaaaaaaat

SEQ ID NO: 20 - DNA, 24 bp, synthetic construct
  misc_feature 1..24: note = Description of Artificial Sequence: primer
  source 1..24: mol_type = other DNA
taacccgtctcctgcaaaatcacg

SEQ ID NO: 21 - DNA, 44 bp, synthetic construct
  misc_feature 1..44: note = Description of Artificial Sequence: primer
  source 1..44: mol_type = other DNA
cgcttatggccaactgctcctgttttttccctatatgcaaaaga

SEQ ID NO: 22 - DNA, 20 bp, synthetic construct
  misc_feature 1..20: note = Description of Artificial Sequence: primer
  source 1..20: mol_type = other DNA
agttggccataagcgcagcg

SEQ ID NO: 23 - DNA, 41 bp, synthetic construct
  misc_feature 1..41: note = Description of Artificial Sequence: primer
  source 1..41: mol_type = other DNA
ttaaacacagataaacttgttcaagctttgatgaaaattgc

SEQ ID NO: 24 - DNA, 27 bp, synthetic construct
  misc_feature 1..27: note = Description of Artificial Sequence: primer
  source 1..27: mol_type = other DNA
tttatctgtgtttaataacggtaaatt

SEQ ID NO: 25 - DNA, 45 bp, synthetic construct
  misc_feature 1..45: note = Description of Artificial Sequence: primer
  source 1..45: mol_type = other DNA
gatattaaagaaactttaatgttaaaacctgaaggctttgtggta

SEQ ID NO: 26 - DNA, 27 bp, synthetic construct
  misc_feature 1..27: note = Description of Artificial Sequence: primer
  source 1..27: mol_type = other DNA
agtttctttaatatccagctcgtagtt